(19) United States
(12) Patent Application Publication
(10) Pub. No.: US 2010/0129370 A1
(43) Pub. Date: May 27, 2010

(54) Trefoil Factors and Methods of Treating Proliferation Disorders Using Same

(76) Inventor: Peter E. Lobie, Auckland (NZ)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/651,850

(22) Filed: Jan. 4, 2010

Related U.S. Application Data
(62) Division of application No. 11/794,025, filed on Apr. 28, 2008.

(30) Foreign Application Priority Data
Dec. 22, 2004 (AU) 2004907226
Dec. 22, 2004 (AU) 2004907262
Dec. 22, 2004 (AU) 2004907263

(51) Int. Cl. A61K 39/395; A61P 35/00

(52) U.S. Cl. 424/138.1

(57) ABSTRACT

The present invention relates to methods of regulation of cellular proliferation and/or survival, particularly methods for the treatment of proliferative disorders. The invention also relates to agents and compositions of use in such methods.

### RELATED APPLICATIONS

This application is a divisional application of U.S. patent application Ser. No. 11/794,025, deposited with the U.S. Patent and Trademark Office on Jun. 21, 2007 as a national stage application filed under 35 U.S.C. §371 of International Application No. PCT/US2005/046634, which was filed on Dec. 22, 2005 and which claims the benefit of Australian Patent Application 2004907226, filed Dec. 22, 2004, Australian Patent Application 2004907262, filed Dec. 22, 2004 and Australian Patent Application 2004907263, filed Dec. 22, 2004, the contents of each of which are hereby incorporated by reference.

### BACKGROUND OF THE INVENTION

The regulation and control of proliferation and/or survival of cells in animals is a complex process involving a number of cellular factors and their interactions with one another. Mutations or alteration in expression in any number of these cellular factors can result in uncontrolled proliferation or growth of cells and ultimately lead to the development of tumors and cancer.

Hormones and/or growth factors are involved in the normal regulation and control of cellular growth and development. For example, growth hormone (GH) is involved in normal pubertal mammary gland development (Walden et al., Endocrinology 139, 659-662, 1998; Kleinberg, J. Mammary Gland Biol. Neoplasia. 2, 49-57, 1997; Bchini et al., (1991) Endocrinology 128, 539-546, 1991; Tornell et al., Int. J. Cancer 49, 114-11, 1991; Nagasawa et al., Eur. J. Cancer Clin. Oncol. 21, 1547-1551, 1985; Swanson and Unterman, Carcinogenesis 23, 977-982, 2002; Stavrou and Kleinberg, Endocrinol. Metab. Clin. North Am. 30, 545-563, 2001; Okada and Kopchick, Trends Mol. Med. 7, 126-132, 2001; Ng et al., Nat. Med. 3, 1141-1144, 1997) and expressed in normal human mammary gland (Raccurt et al., J. Endocrinol. 175, 307-318, 2002). Alterations in expression levels of hormones such as GH can result in aberrant proliferation of cells. For example, increased epithelial expression of the hGH gene is associated with the acquisition of pathological proliferation, and the highest level of hGH gene expression is observed in metastatic mammary carcinoma cells (Raccurt et al., J. Endocrinol. 175, 307-318, 2002). Such alterations in expression of autocrine hGH may result in the transformation of a normal cell to a cancer cell.

There is a need to understand further the effects of hormones and/or growth factors on the development of proliferative disorders, including identifying any cellular factors which promote cell proliferation, cell survival and/or oncogenic transformation. This will aid in the identification of means for the regulation of proliferation and/or survival, and particularly means for the treatment of proliferative disorders such as cancer.

### SUMMARY OF THE INVENTION

The invention provides methods of inhibiting the proliferation of one or more tumor cells by decreasing the production of a trefoil factor (TFF) gene product or an activity of such a gene product in a tumor cell. Tumors to be inhibited in such a manner include tumors characterized as expressing increased levels of TFF compared to normal, noncancerous cells. Examples of such tumor cells include drug-resistant tumors such as anti-estrogen therapy-resistant tumors and hormone-sensitive tumor cells such estrogen-sensitive breast cancers (e.g., tumors of mammary glands) and other tumors of reproductive organs such ovaries, cervix, prostate; gastro-intestinal tract tumors (colon, stomach, liver, esophagus, pancreas); and any other tumors where TFF is important for cell proliferation, survival and oncogenicity. Other hormone-sensitive tumors include androgen-sensitive tumors, GH-sensitive tumors, prolactin sensitive tumors, progesterone sensitive tumors. In addition to TFF-overexpressing tumors, the methods of the invention are applicable to tumors where TFF is important for cell proliferation and survival (TFF-dependent tumors). TFF-overexpressing tumors are identified using standard methods of determining protein or transcript levels, e.g., ELISA, reverse transcriptase-polymerase chain reaction (RT-PCR), immunohistochemistry, compared to normal non-tumor cells; TFF-dependent tumors are identified by detection of a decrease in proliferation/survival in the presence of a TFF inhibitor (e.g., peptide antagonist, TFF-specific antibody) compared to that in its absence; and hormone-sensitive tumors are identified by detection of a decrease in proliferation/survival in the presence of a hormone inhibitor (e.g., hormone-specific antagonist, antibody) compared to that in its absence.

The method includes a step of identifying a subject with a tumor expressing an increased level of TFF, a tamoxifen-resistant tumor or a hormone-sensitive tumor such as an estrogen-receptor positive breast cancer and administering to the subject a TFF inhibitory compound. The identifying step is carried out by detecting an increase in the level of TFF1, TFF2, or TFF3 expression compared to a normal control level (e.g., a level of TFF in a subject or pool of normal, healthy, without evidence of cancer). TFF-specific antibodies, e.g., antibodies that specifically bind to TFF1 and/or TFF3 are used to distinguish tamoxifen-resistant tumor cells from other tumor cells, because TFF over expression is an identifying feature of drug resistant tumors. Such antibodies are also useful alone and together to inhibit drug resistance, i.e. increase drug (e.g. tamoxifen) sensitivity. Thus, the antibodies are optionally co-administered with a chemotherapeutic agent such as anti-estrogen (tamoxifen or aromatase inhibitors), anti-androgens and other anti-neoplastic agents described herein.

TFF-specific antibodies are useful to reverse tamoxifen resistance and to increase endocrine sensitivity of tumors. Accordingly, a method of increasing sensitivity of a drug-resistant tumor to a chemotherapeutic agent is carried out by contacting a drug-resistant tumor with an antibody composition that binds to a TFF gene product such as TFF1, 2, and/or 3. Preferably, the antibody composition contains a mixture of antibodies such as an antibody that binds to TFF1 and an antibody that binds to TFF3. In the latter case, the effect of a TFF1 and TFF3 antibodies is a synergistic effect. In some examples, the tumor is resistant to tamoxifen such as a drug-resistant breast carcinoma; in such cases, the antibodies are co-administered with the drug such as tamoxifen. In the case of hormone-sensitive tumors, the antibodies are optionally co-administered with a hormone antagonist such as an anti-estrogen compound. In co-administration regimens, the antibody composition and chemotherapeutic agent are provided simultaneously or sequentially.

TFF specific antibodies include those that bind to domains or residues that are exposed (e.g., outer loop structure residues in the tertiary structure of the protein in solution, participate in TFF dimerization, aggregation, as well as domains responsible for promoting cellular proliferation, survival, and oncogenicity. For example, the epitope binding specificity of the antibody includes a TFF sequence that contains a domain involved in stimulation of cell proliferation, survival and oncogenicity. For example, an antibody binds to an epitope containing residue 24, 25, 26, 47, or 57 of the mature form of TFF3; residue 20, 21, 42, 43, or 58 of TFF1; or residue 21, 22, 43, 44, 70, 71, 92, 93 or 103 of TFF2. The antibody is a polyclonal antisera or monoclonal antibody or derivative of either of those. The invention encompasses not only an intact monoclonal antibody, but also an immunologically-active antibody fragment, e.g., a Fab or (Fab)₂ fragment; an engineered single chain Fv molecule; or a chimeric molecule, e.g., an antibody which contains the binding specificity of one antibody, e.g., of murine origin, and the remaining portions of another antibody, e.g., of human origin. Other TFF-binding antibodies are used to directly target TFF-over-expressing cells for destruction. In the latter case, the antibody, or fragment thereof, activates complement in a patient treated with the antibody. Preferably, the antibody mediates antibody-dependent cytotoxicity of tumor cells in the patient treated with the antibody. The antibody, or fragment thereof, is administered alone or conjugated to a cytotoxic agent. Binding of the antibody to a tumor cell expressing TFF results in impairment or death of the cell, thereby reducing tumor load. The antibody is optionally conjugated to a radiochemical, or a chemical tag which sensitizes the cell to which it is bound to radiation or laser-mediated killing.

The present invention also provides methods of inhibiting proliferation of a tumor cell, by contacting the cell with a compound that inhibits the expression of a trefoil factor (TFF) gene, such as TFF1 or TFF2. For example, the compound includes one or more iRNAs or one or more DNA molecules encoding one or more iRNAs, and the expressed iRNAs interfere with the mRNA of the TFF1, TFF2 genes thereby inhibiting expression of the TFF1 or TFF2 genes, which in turn inhibits proliferation of the cell.

Inhibition of tumor cell proliferation is also carried out by contacting the cell with a compound that inhibits the expression of a trefoil factor 3 (TFF3) gene, the compound comprising one or more iRNAs containing a nucleotide sequence selected from the group consisting of SEQ ID NO: 3-11 or one or more DNA molecules encoding one or more iRNAs containing the nucleotide sequence selected from the group consisting of SEQ ID NO: 3-11, thereby inhibiting expression of the TFF3 gene and concomitantly proliferation of the cell.

The present invention also provides methods of inhibiting proliferation or inhibiting/preventing access of TFF to a tumor cell, by contacting the cell with a peptide antagonist of a TFF or prevent access of TFF to the cell. The antagonist is selected from the group consisting of (a) a mutant of a TFF of SEQ ID NO:25 with a non-identical amino acid at positions 23, 24, 25, 46, 47 or 57; (b) a fragment of the TFF of SEQ ID NO:25 or c) a chimera of a whole or fragment or mutant of the TFF of SEQ ID NO: 25 fused with another protein of interest (e.g., a protein other than a TFF protein such as human serum albumin protein, beta casein). The antagonist inhibits binding of an endogenous TFF to a TFF receptor; prevents or inhibits aggregation of the TFF receptor in the cell or inhibits association of TFF polypeptides, e.g., TFF dimerization or aggregation. The TFF mutant preferably inhibits a function of endogenous TFF such as oncogenicity and/or potentiation of tumor cell proliferation. TFF antagonists also include TFF1 and TFF2 mutants in which the residues located at corresponding residues in the structure of TFF1 and TFF2, respectively, are substituted with a non-identical amino acid relative to the wild type sequence.

The invention includes methods of diagnosing a anti-estrogen-therapy resistance (e.g., tamoxifen-resistance) or a predisposition to developing the resistance by detecting the level of TFF1, 2, or 3 expression in a subject-derived tissue sample, wherein an increase in the level compared to a normal control level indicates that the subject comprises a tamoxifen-resistant tumor or is at risk of developing the tumor. Also included are methods of reducing tamoxifen resistance by inhibiting TFF expression activity, or dimerization, e.g. by contacting a resistant cell with a TFF-specific antibody or other TFF binding ligand.

Methods of treating or preventing cancer or a cell proliferation disorder in a subject in need thereof, involve regulating the expression of a trefoil factor (TFF) gene, such as TFF1, TFF2 or TFF3, by administering a composition comprising one or more iRNAs or one or more DNA molecules encoding one or more iRNAs, that interfere with the mRNA of the TFF1, 2, or 3 gene and decrease expression of the TFF1, TFF2, or TFF3 gene product. One or more peptide antagonists or one or more DNA molecules encoding one or more peptide antagonists are useful to inhibit an activity e.g., functional activity of the TFF protein such as oncogenicity or induction of cell proliferation or survival. The peptide antagonist can be selected from the group consisting of (a) a function interfering mutant of a TFF having a sequence SEQ ID NO:25 modified to contain a non-identical amino acid at positions 23, 24, 25, 46, 47 or 57; (b) a fragment of a TFF of SEQ ID NO:25 or chimera of a whole or a fragment or mutant of the TFF of SEQ ID NO: 25 fused with a protein of interest (e.g. human serum albumin protein, beta casein). Other antagonists include TFF1 and TFF2 mutants with corresponding alterations in protein sequence.

TFF1 peptide antagonists include TFF1 mutants in which the mutant contains a non-identical amino acid at position 20, 21, 42, 43, and/or 58 of SEQ ID NO:36 (TFF1); TFF2a peptide antagonists include TFF2a mutants in which the mutant comprising a non-identical amino acid at position 21, 22, 43, and/or 44 of SEQ ID NO:37 (TFF2a), and; TFF2a peptide antagonists include TFF2a mutants in which the mutant contains a non-identical amino acid at position 70, 71, 92, 93, and/or 103 of SEQ ID NO:37 (TFF2b). These mutant TFF polypeptides are the same length or shorter than the corresponding wild type full length molecule and are optionally linked, e.g., conjugated to or produced as a recombinant chimeric protein, to a heterologous polypeptide such as human serum albumin or beta-casein. Peptide antagonists also include TFF fragments that are optionally linked to a heterologous peptide as described above. Preferably, the TFF fragments are 10, 20, 30, 40, 50, 60, 75, 100 amino acids in length or any length that contains few amino acids compared to full length mature TFF protein. The fragments contain consecutive amino acids of a native wild type TFF sequence and are purified from sequences which naturally flank the fragment in the wild type protein. Optionally, the fragments are TFF mutants that contain one, two, or three amino acid substitutions relative to the native sequence.

The antagonist inhibits binding of an endogenous TFF to a TFF receptor in the cell or inhibits TFF dimerization or inhibits TFF function. A peptide TFF antagonist compound is of sufficient molecular mass such rapid excretion by the kidneys is minimized. For example, a TFF binding composition is associated with, e.g., fused to another compound such as another peptide (e.g. human serum albumin, beta casein or other suitable protein) to increase the molecular mass of the composite compound. Preferably, the molecular mass of the antagonist is sufficient to inhibit access of endogenous TFF to a tumor cell. For example, the antagonist is a chimeric protein composition that contains a mutant TFF sequence and a non-TFF peptide sequence, and the molecular mass of the antagonist compound is greater than 2, 3, 5, 10, 25, 35, 45, 55, or 65 kDa.

Optionally, the compositions contain a pharmaceutically acceptable carrier or a second compound. For example, the second compound is a anti-estrogen therapeutic, chemotherapeutic or anti-neoplastic agent, e.g., tamoxifen. Such agents are administered sequentially (prior to or after the antagonist) or simultaneously.

The tumor or cancer is selected from the group consisting of lung cancer, colorectal cancer, breast cancer, pancreatic cancer, ovarian cancer, prostate cancer, renal carcinoma, hepatoma, brain cancer, melanoma, multiple myeloma, hematologic tumor, and lymphoid tumor. Preferably, the cancer is an endocrine sensitive cancer.

Proliferative disorders include keratinocyte hyperproliferation, inflammatory cell infiltration, cytokine alteration, epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses.

The subject is a mammal, preferably a human suffering from or at risk of developing a tumor characterized by an aberrant level of a TFF gene product. The compositions and methods are also useful for veterinary use, e.g., in treating, cats, dogs, and other pets in addition to livestock, horses, cattle and the like.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1A is a photograph of a blot demonstrating forced expression of TFF3 mRNA upon stable transfection of MCF-7 cells with TFF3 cDNA. MCF-7 cells were stably transfected with either the empty vector (MCF7-VECTOR) or the vector containing TFF3 cDNA (MCF7-TFF3). The level of TFF3 mRNA was determined by RT-PCR under serum free conditions. FIG. 1B is Western blot showing increased TFF3 peptide production by MCF-7-TFF3 cells compared to the vector transfected control. The vector-transfected group shows endogenous TFF3 levels in these cells. β-Actin was used as loading control.

FIGS. 2A-C are bar graphs and FIG. 2D is a linear graph showing the effect of forced expression of TFF3 in mammary carcinoma cells on cell cycle progression (5′-bromo-2′-deoxyuridine incorporation), apoptosis/cell survival and total cell number. MCF7-VECTOR and MCF7-TFF3 cells were cultured in serum-free media or in media supplemented with 10% FBS as indicated. Cell cycle progression (BrdUrd incorporation) (A), apoptotic cell death (B), a total cell number (C) and total cell number over a period of 10 days (D) were determined in both cell lines under the indicated conditions.

FIGS. 3A-B are bar graphs showing the effect of forced expression of TFF3 on soft agar colony formation. Soft agar colony formation by MCF-7 cells transiently (A) or stably transfected with TFF3 cDNA (B). Increased expression of TFF3 results in enhancement of soft agar colony formation indicating TFF3 favours oncogenic transformation. The results are given as means±S.D. of triplicate experiments.

FIG. 4A is a line graph showing the effect of expression of TFF3 on mammary carcinoma cell proliferation in suspension culture. FIG. 4B is a graph and photograph of cells showing the effect of expression of TFF3 on foci formation. The results are given as means±S.D. of triplicate experiments.

FIG. 5A is a photograph of an electrophoretic gel and FIG. 5B is a bar graph showing the effect of inhibition of TFF3 expression on soft agar colony formation in human mammary carcinoma cells (MCF-7). MCF-7 cells were transiently transfected with either an empty vector or a vector producing TFF3 RNAi. (A) Effect of siRNA on the level of TFF3 mRNA in MCF-7 cells. (B) Soft agar colony formation in MCF-7 cells transfected with siRNA to TFF3. The results are given as means±S.D. of triplicate experiments.

FIG. 6 is a bar graph showing forced expression of TFF3 in immortalised human epithelial cell line MCF10A results in colony formation in soft agar indicative of oncogenic transformation. Transfection of MCF-10A cells with an empty vector does not result in oncogenic transformation. The results are given as means±S.D. of triplicate experiments.

FIG. 7A is a photograph of a blot showing expression of TFF3 mRNA upon incubation with estrogen/estradiol (E2) and tamoxifen in MCF-7 cells. FIG. 7B is a bar graph showing an increase in TFF3 promoter activity following incubation with estrogen/estradiol (E2) and tamoxifen in MCF-7 cells.

FIG. 8 is a bar graph showing forced expression of TFF3 in human mammary carcinoma results in tamoxifen resistance. Soft agar colony formation by MCF-7 cells transfected by vector and MCF-7 cells transfected with TFF3 cDNA was determined in the presence and absence of tamoxifen. MCF-7 cells with forced expression of TFF3 were resistant to the inhibitory effects of tamoxifen on soft agar colony formation.

FIG. 9A is a photograph of a blot showing that estrogen and tamoxifen treatment increase the transcriptional levels of TFF1 in MCF-7 cells. FIG. 9B is a graph showing an increase in TFF1 promoter activity in tamoxifen-resistant MCF-7 cells.

FIG. 10A is a graph showing total cell number of MCF-7 cells with or without TFF3 following estrogen treatment. FIG. 10B is a graph and photograph of cells showing that TFF3 increases total colony number in the presence of estrogen.

FIG. 11A is a graph showing an increase in TFF3 promoter activity in tamoxifen-resistant MCF-7 cells. FIG. 11B is a photograph of a blot showing that TFF3 mRNA is over-expressed in tamoxifen-resistant MCF-7 cells.

FIG. 12A is a graph showing that that knockdown of TFF3 using siRNA increases tamoxifen sensitivity in tamoxifen-resistant MCF-7 cells. FIG. 12B is a graph showing inhibition effects of TFF1 and TFF3 siRNA, given alone or in combination, on colony formation of MCF-7 cells on soft agar.

FIG. 13 is a bar graph showing inhibitory effects of TFF3 and human serum albumin (hSA) fusion protein on TFF3 biological activities. The effects of TFF3 and its mutants on the transcription of CEACAM6 gene. MCF-7 cells were co-transfected with CEACAM6 luciferase report plasmid as well as expression plasmids of TFF3 and TFF3-C57F mutant, has and TFF3-C57F-has fusion protein by Saint Mix transfection reagent. Luciferase activity was measured. Experiments were carried out in 6-well palates in three replicates.

FIG. 14 is a graph showing a growth curve for MCF-7 cells transfected with TFF3 mutants.

FIG. 15A-B are photographs of an immunoblot showing the expression of recombinant TFF1 (A) and TFF3 (B) proteins in bacteria.

FIGS. 16A-B are bar graphs showing inhibition of proliferation of MCF-7 cells following 48 hr (A) or 72 hr (B) incubation with anti-TFF polyclonal antibodies. MCF-7 cells were seeded into 96 wells microplates with affinity purified rabbit anti-TFF1 or TFF3 antibodies at a concentration of 0, 1, 5 and 20 μg/ml of each antibody or their pre-immune sera as controls. The cells were incubated for 48 h (A) or 72 h (B) and cell proliferation was determined by MTT assay using the procedure described in Material and Methods. CK-06, -07, -08 and -09 are the pre-immune sera for the rabbit polyclonal antibodies of human TFF1 (anti-F1-06 and -07) and TFF3 (anti-F3-08 and -09), respectively.

FIG. 16C shows inhibition of MCF-7 cell proliferation by rabbit anti-TFF1 or TFF3 polyclonal antibodies alone or in combination.

FIG. 17 is a bar graph and FIG. 17B is a series of photographs of immunostained MCF-7 cells showing the prevention of colony formation in soft agar following incubation with anti-TFF polyclonal antibodies.

FIG. 18 is a bar graph showing inhibition of proliferation of tamoxifen-resistant MCF-7 cells following incubation with anti-TFF polyclonal antibodies.

FIG. 19 is series of photographs of immunostained MCF-7 cells showing the effect of anti-TFF polyclonal antibodies on tamoxifen-resistant MCF-7 cell morphology.

### DETAILED DESCRIPTION OF THE INVENTION

The trefoil factor family of proteins are characterised by a 40-amino acid trefoil motif that contains 3 conserved disulfide bonds. The 3 intrachain disulfide bonds form the trefoil motif (TFF domain). The trefoil motif is known in the art, e.g. Taupin and Podolsky, Nat Rev Mol Cell Bio. 4(9):721-32, 2003; Hoffmann et al., Histol Histopathol 16(1):319-34, 2001; and Thim, Cell Mol Life Sci 53(11-12):888-903, 1997.

In humans, three distinct members of the trefoil peptides have been identified. TFF1 or pS2 was first detected in a mammary cancer cell line as an estrogen-inducible gene. In human stomach, it is predominantly located in the foveolar cells of the gastric mucosa. TFF2 (formerly spasmolytic polypeptide or SP) was first purified from porcine pancreas and is expressed in mucous neck cells, deep pyloric glands, and Brunner's glands. TFF3 or intestinal trefoil factor (ITF) was the last to be identified and is predominantly expressed in the goblet cells of the small and large intestine. The trefoil peptides are involved in mucosal healing processes and are expressed at abnormal elevated levels in neoplastic diseases. A wide range of human carcinomas and gastrointestinal inflammatory malignancies, including peptic ulceration and colitis, Crohn's syndrome, pancreatitis, and biliary disease, aberrantly express trefoil peptides. Orthologues of these human proteins have been identified in other animals; for example, rats, mice and primates.

The trefoil family of peptides possess divergent function in the mammary gland with TFF1 functioning as a mitogen and TFF2 stimulating branching morphogenesis and cell survival. TFF3 is widely co-expressed with TFF1 in malignancies of the human mammary gland whereas TFF2 is not expressed in the mammary epithelial cells. Although there is some diversity of function and expression among the trefoil family of peptides, the results of the instant invention indicate that modulation (e.g., inhibition) of any member of the TFF family (i.e., TFF1, TFF2 or TFF3) has similar benefits.

Reference herein to “TFF”, “TFF protein(s)”, or “TFF family of proteins” refers to the group of related proteins including TFF1, TFF2, and TFF3. TFF proteins share at least approximately 28 to 45% amino acid identity within the same species.

Functional analysis of TFF3 determined that its expression is sufficient to stimulate an increase in total cell number by concomitant increase in mitogenesis and cell survival, support anchorage independent growth of human carcinoma cells and other indices of oncogenicity including growth in suspension culture and foci formation. Furthermore, TFF supported oncogenic transformation of immortalized, but otherwise normal, human epithelial cells. siRNA mediated decrease of TFF3 expression concordantly abrogated anchorage independent growth of human carcinoma cells. These results indicate that inhibition of TFF expression and/or activity leads to a decrease in cellular proliferation and decreases the progression and severity of cancer and other proliferative disorders. Similar results can be shown with other members of the TFF family of proteins.

The data described herein were generated using the following materials and methods.

Cell Culture

MCF-7 (HTB-22) and MCF-10A (CRL-10317) cell lines were obtained from the ATCC. MCF-7 cells (Kaulsay et al., Exp. Cell Res. 250:35-50, 1999) were cultured at 37° C. in 5% CO² in RPMI supplemented with 10% heat inactivated fetal bovine serum (FBS), 100 units/ml penicillin, 100 μg/ml streptomycin, and 2 mM L-glutamine. The original MCF7 cells are tamoxifen sensitive and cannot grow in tamoxifen-containing medium. After having been progressively exposed to increasing concentrations of tamoxifen (Sigma Chemical Co., St. Louis, Mo.), the MCF-7 cells have adapted themselves to grow in media containing up to 1 μM of tamoxifen. These tamoxifen-resistant MCF-7 cells (termed MCF-7-Tam^{R}) have been maintained in media containing 1 μM of tamoxifen for more than six months before starting experiments. The tamoxifen resistance of MCF-7-Tam^{R} cells was established by comparing with parental MCF7 cells cultured in the same media but without tamoxifen.

MCF-10A cells and derivatives were cultured in Dulbecco's modified Eagle's medium/F-12 medium (Invitrogen, Carlsbad, Calif.) supplemented with 5% horse serum (Invitrogen) plus 2 mM glutamine, 100 μg/ml streptomycin, 100 IU/ml penicillin, 0.25 μg/ml ampicillin B, 100 ng/ml cholera toxin, 20 ng/ml epidermal growth factor (Upstate Biotechnology, Lake Placid, N.Y.), 0.5 μg/ml hydrocortisone (Calbiochem, La Jolla, Calif.), and 10 μg/ml insulin.

### Preparation of Total RNA

Total RNA was isolated from MCF-7 cells using TRI-REAGENT according to the manufacturer's instructions and resuspended in diethyl pyrocarbonate-treated water. Quantification and purity of the RNA was assessed by A260/A280 absorption, and RNA quality was assessed by agarose gel electrophoresis. Three independently derived total RNA samples from the respective cell lines were pooled before labeling for hybridization. RNA samples with ratios greater than 1.6 were stored at −80° C. for further analysis.

### RT-PCR and Primers

RT-PCR was performed in a final volume of 50 ml containing 0.2 μg of mRNA template, 0.6 μM primers, 2 ml of enzyme mix, 400 μM of each dNTP, 10 reaction buffer, and 10 Q-Solution by use of the Qiagen One-step RT-PCR kit. RNA template was reverse-transcribed into cDNA for 30 min at 50° C.; Hotstart TaqDNA polymerase was activated by heating for 15 min at 95° C.; the denatured cDNA templates were amplified by the following cycles: 94° C./30 s, 55° C./30 s, and 72° C./60 s. A final extension was performed for 10 min at 72° C. In order to compare the PCR products semiquantitatively, 15-40 cycles of PCR (annealing temperature 55° C.) were performed to determine the linearity of the PCR amplification, and the amplified β-actin cDNA served as an internal control for cDNA quantity and quality. All RNA samples were treated with DNase I to avoid genomic DNA contaminations.

| | |
|---|---|
| TFF3 (sense) | |
| 5′-CTGAGGCAC CTCCAGCTGC CCCCG-3′ | (SEQ ID NO: 26) |
| TFF3 (antisense) | |
| 5′-GGAGCATGGGACCTTTATTCG-3′ | (SEQ ID NO: 27) |

Amplified PCR products were visualized on a 1% agarose gel. Amplification yielded the predicted size of the respective amplified fragments.

### Cloning of Human TFF3:

To clone human TFF3 cDNA, total RNA was extracted from MCF-7 cells using TRI-REAGENT and the cDNA was amplified using the primers TFF3 cDNA Top 5′ CTC TGC ATG CTG GGG CTG GTC 3′ (SEQ ID NO:28); Bot 5′ GGA GGT GCC TCA GAA GGT GCA TTC 3′ (SEQ ID NO:29) and cloned in to PCR-script™ AmpSK⁺ vector. TFF3 cDNA inserts were reamplified with primers TFF3 cDNA-myc Top 5′ GCG AAG CTT ATG CTG GGG CTG GTC 3′ (SEQ ID NO:30); TFF3 cDNA-myc Bot 5′ GGA GGT CCG CGG GAA GGT GCA TTC 3′ (SEQ ID NO:31) and cloned in frame using the enzyme sites HindIII and SacII inserted in the primers in pcDNA3.1/Myc-His-B vector and the sequence verified. Expression of TFF3 was verified by RT-PCR and western blot analysis.

Construction of siRNA to Human TFF3:

A human TFF3 RNAi construct in pRNA-U6.1/Hygro vector from Genscript was generated targeting the sequence ACTAGGAAGACAGAATGCA (SEQ ID NO:32). 5×10⁴ MCF-7 cells were seeded into six-well plates and were cultured as above. They were transiently transfected with 1 μg of the RNAi constructs or the pRNA-U6.1/Hygro vector and grown for additional 18 hrs.

### Recombinant TFF1 and TFF3 Proteins

The Glutathione S-transferase (GST) Gene Fusion System from Amersham Biosciences was used to produce recombinant TFF 1 and TFF3 proteins in E. coli bacteria. The full-length human TFF1 and TFF3 cDNA fragments coding for mature proteins were amplified using RT-PCR from MCF-7 cells. The RT-PCR products were cloned into the pGEX 4T1 vector (Amersham Biosciences) upon 5′ EcoR I and 3′ Xho I digestion to generate pGEX 4T1-TFF1 and pGEX 4T1-TFF3 plasmids for the expression of GST fusion proteins in E. coli. The sequences of the plasmids were verified by DNA sequencing.

The pGEX 4T1-TFF1 or pGEX 4T1-TFF3 plasmid was used to transform BL21-Gold cells (Stratagene). A single recombinant E. coli colony was inoculated into LB medium containing carbenicillin (50 μg/ml). The overnight culture was diluted 1:200 in LB medium/carbenicillin, pH 7.4, and cultured at 37° C. to optical density at 600 nm of ˜0.5. Protein expression was then induced by adding isopropyl-β-D-thiogalactopyranoside (IPTG) to a final concentration of 0.2 mM and the cultures were incubated for an additional 3-4 hours at 37° C. Cells were harvested and GST-fusion proteins were extracted from bacterial cells under non-denaturing conditions. Proteins were isolated using a standard purification method for efficient solubilisation and were subsequently bound with high affinity to glutathione sepharose 4B matrix (Amersham Biosciences). The GST fusion protein bound to the column was digested with thrombin to cleave TFF1 or TFF3 proteins from GST and eluted from column with PBS to yield essentially pure product. The integrity of the GST fusion proteins and purified TFF1 and TFF3 proteins were analysed by SDS-polyacrylamide gel electrophoresis (PAGE) on a 4-12% NuPAGE Bis-tris gel (Invitrogen) and visualised by Coomassie Blue staining. The yields of purified fusion proteins were estimated by Bradford's Assay.

### Antibody Production

Purified recombinant TFF1 and TFF3 proteins produced in E. coli were used to raise rabbit anti-TFF1 and -TFF3 polyclonal antibodies by Biogene, Germany. Two rabbits were immunized for each protein. The antibodies were affinity purified from the antisera. Antibodies produced recognized and bound to TFF proteins in their native tertiary structure in solution. Epitope specificity is defined by tertiary structure (e.g., exposed residues in the 3-dimensional structure of TFF).

### MTT Proliferation Assay

MCF-7 cells were seeded at a concentration of 5×10³ cells/well in 100 μl RPMI-1640 culture medium containing 10% FBS into 96 well flat bottom tissue culture microplates with various amounts (0, 1, 5 and 20 μg/ml) of the affinity purified rabbit anti human TFF1 or TFF3 antibodies as well as their pre-immune sera as controls. The cells were incubated for 2 or 3 days in a humidified incubator with 5% CO₂ at 37° C. After the incubation period, 10 μl of the MTT (3-[4,5-dimethylthiazol-2-yl]-2,5-diphenyl tetrazolium bromide) labelling reagent was added at a final concentration 0.5 mg/ml to each well and the cells were labelled for 5 h in the incubator. At the end of the incubation period, the medium were removed and the converted dye was solubilized with 100 μl solubilizing solution (0.01N HCl in 10% SDS in water). The plates were then sealed and placed at 37° C. overnight to completely solubilize the purple formazan crystals. The absorbance of the converted dye was measured using Spectra Max 250 multiplate reader at a wavelength of 570 nm with background subtraction at 650 nm.

### Cell Morphology Assay

Tamoxifen resistant MCF-7-Tam^{R} cells were plated at a concentration of 5×10³ cells/well in 96-well plates in 100 μl of phenol red free RPMI 1640 medium containing dextran coated charcoal stripped 10% FBS. The plates were incubated at 37° C. for 24 h to allow the cells to reattach and re-equilibrate. Without removing any medium, anti-TFF3 antibody and/or tamoxifen were then added at twice the final concentration in a volume of 100 μl of the culture media. After 48 h images of cells were taken at 10× magnification under phase contrast microscope (Olympus IX70).

5′-Bromo-2′-deoxyuridine Incorporation Assay and Measurement of Apoptosis Mitogenesis was directly assayed by measuring the incorporation of BrdUrd. For incorporation of BrdUrd, subconfluent cells were pulse-labeled with 20 mM BrdUrd for 30 min, washed twice with PBS, and fixed in cold 70% ethanol for 30 min. BrdUrd detection was performed by use of the BrdUrd staining kit from Zymed Laboratories Inc. (San Francisco, Calif.) according to the manufacturer's instructions. A total population of over three times 300 cells was analyzed in several arbitrarily chosen microscopic fields to determine the BrdUrd labeling index (percentage of cells synthesizing DNA).

Apoptotic cell death was measured by fluorescent microscopic analysis of cell DNA staining patterns with Hoechst 33258 from Sigma Chemical Co. (St Louis, Mo.) (Garverick et al., Reproduction 123:651-61, 2002). Cells were trypsinized with 0.5% trypsin and washed twice with serum-free medium. The cells were then seeded to glass cover slips in six-well plates and incubated in serum-free medium. After a culture period of 24 h, the cells were fixed in 4% paraformaldehyde in PBS (pH 7.4) and stained with the karyophilic dye Hoechst 33258 (20 g/ml) for 10 min at room temperature. Following washing with PBS, nuclear morphology was examined under a UV-visible fluorescence microscope (Zeiss Axioplan). Apoptotic cells were distinguished from viable cells by their nuclear morphology characterized by nuclear condensation and fragmentation as well as the higher intensity of the blue fluorescence of the nuclei. For statistical analysis, three times 300 cells were counted in eight random microscopic fields at 400 magnification.

### Soft Agar Colony Formation

To verify the effect of increase in TFF3 activity (5×10⁴) MCF-7 cells were seeded into six-well plates and were cultured as above in RPMI medium. They were transiently transfected with 1 μg of the TFF3-cDNA construct or the pcDNA3.1/Myc-His-B vector and grown for additional 18 hrs. For soft-agar colony formation assay six well plates were first covered with an agar layer (RPMI 1640 with 0.5% agar and 10% FBS). The upper layer contains 5×10³ cells in RPMI 1640 with 0.35% agar and 10% FBS. Cells transfected with TFF3-cDNA or the pcDNA3.1/Myc-His-B were trypsinised and used verify the effect of increase in TFF3 and TFF3 RNAi or the vector pRNA-U6.1/Hygro transfected cells were seeded as above in the experiment to verify the effect of decrease in TFF3 in colony formation. Medium was added to prevent drying. The plates were incubated for 9 days (for MCF-7 cells) or 14 days (for MCF-10A cells), after which the cultures were inspected and photographed. To assess MCF10A-vector and MCF10A-TFF3 cell soft agar colony formation assay was performed as above but in Dulbecco's modified Eagle's medium/F12. The colonies in the plates were counted after incubating them at 37° C. for 9 days (MCF-7 cells) and 14 days (MCF-10A cells) and the relative number of colonies calculated. For tamoxifen treatment of MCF-7 cells, 1 μM tamoxifen was added to the agar and media layers and was present for the duration of the experiment.

### Suspension Culture Assay

Cells (5×10⁴) in suspension culture were grown in 30-mm plastic bacteriological dishes (Sterilin, Teddington, United Kingdom). Following culture, cells were harvested and counted.

Foci formation Assay

5000 single cells of MCF7-VEC or MCF7-TFF3 from 70% confluent cultures were seeded in a 35 mm dish in triplicates. The cells were cultured for 2 weeks in the complete media with media changes every 3^{rd} day. The cells were fixed using 70% ethanol and later stained with 0.01% crystal violet in PBS for 1 hour and destained in PBS (3 washes). The destaining was continued until discrete foci of transformed cells which stained darkly were visualized at 4× magnification under inverted microscope. Total number of foci formed per 35 mm dish was counted and mean of the triplicates and standard error to the mean calculated.

### Plasmid Constructs

c-Myc episode tagged human TFF3 expression vector pIRESneo3-TFF3-Myc was constructed as follows: the human TFF3 cDNA was amplified by PCR using the primer set, 5′-ATG GCT GCC AGA GCG CTC TGC-3′ (sense) (SEQ ID NO:39) and 5′-AAG GTG CAT TCT GCT TCC TGC AG-3′ (antisense) (SEQ ID NO:40) and cloned into pIRESneo3 vector (Invitrogen). A sequence coding for 2 copies of c-Myc tag EQKLISEEDL (SEQ ID NO:41) was inserted in frame with the last amino acid of TFF3. The C57F mutant expression vector of TFF3, pIRESneo3-TFF3-C57F-Myc, was similarly made by using the same sense primer and an antisense primer of 5′-AAG GTG AAT TCT GCT TCC TGC AGG G-3′ (SEQ ID NO:42).

The human serum albumin cDNA was also similarly cloned into pIRESneo3 vector with 2 copies of the c-Myc-tag at its C-terminal using primers: 5′-ATG AAG TGG GTA ACC TTT ATT TCC-3′ (sense) (SEQ ID NO:43) and 5′-AAG CCT AAG GCA GCT TGA CTT G-3′ (antisense) (SEQ ID NO:44).

The pIRESneo3-TFF3-C57F-hSA-Myc vector was constructed such as the C57F mutant of TFF3 protein was immediately fused to the mature human serum albumin protein followed by 2 copies of the c-Myc tags at the C-terminal.

Luciferase reporter plasmid, pGL3-CEACAM6 was constructed as follows: the CEACAM6 promoter of 1278 base pairs were amplified by PCR with Vent DNA polymerase using human genomic DNA purified from MCF-7 cells as the template using primer pair, 5′-CCA GCA TAG ACA CTC TCT TTG G (sense) (SEQ ID NO:45) and 5′-GGT CTC TGC TGT CTT CTC TGT C-3′ (antisense) (SEQ ID NO:46). The resulting DNA fragments were introduced into Sma I-digested promoterless luciferase reporter pGL3 basic vector (Promega).

### Luciferase Reporter Assay

MCF-7 cells at 60-80% confluence were co-transfected with luciferase report plasmid pGL3-CEACAM6 as well as expression plasmids of TFF3 and TFF3-C57F mutant, hSA and TFF3-C57F-hSA fusion protein by Saint Mix transfection reagent as per the manufacturer's instructions (Syvolux therapeutics). 36 hours post transfection the cells were washed in cold PBS three times and lysed with 200 μl of the 1× lysis buffer by a freeze-thaw cycle, and lysate was collected by centrifugation at 14,000 rpm for 2 min in a bench top centrifuge. Twenty microliters of supernatant was used for the assay of luciferase activity using a kit (Promega). The luciferase activities were normalized for the amount of the protein in cell lysates. Experiments were carried out in three replicates.

### EXAMPLES

### Example 1

### TFF3 Increases Oncogenicity and Stimulates Oncogenic Transformation; Inhibition of TFF Decreases Oncogenicity

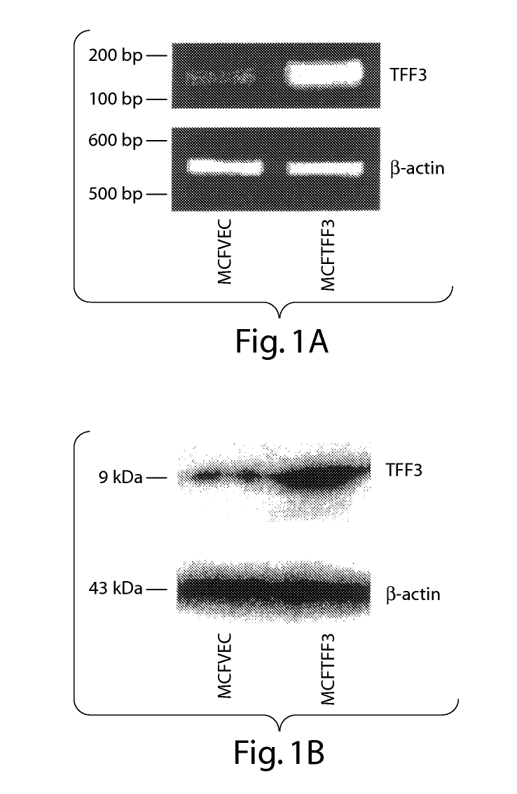

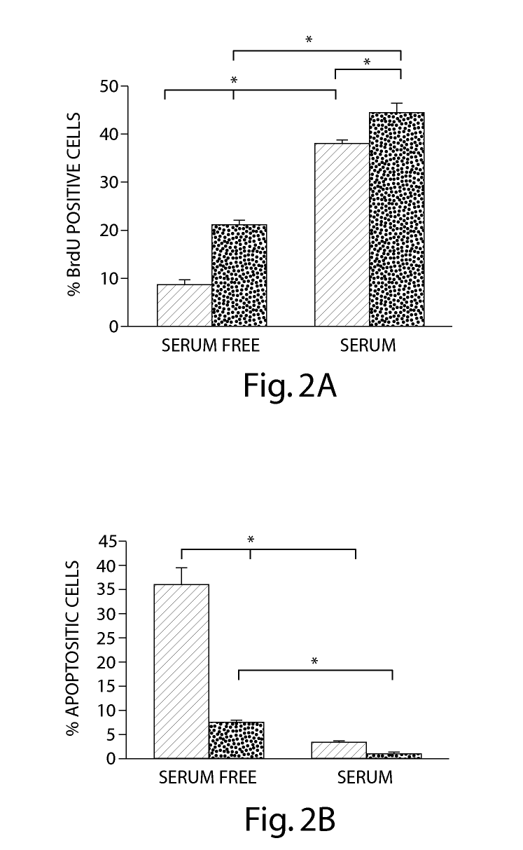

TFF3 increases mammary carcinoma cell mitogenesis and survival and hence cell number. The inventors cloned the complete human TFF3 cDNA and sequence verified the clones. The inventors subsequently generated a human mammary carcinoma (MCF-7) cell line with stable expression of TFF3 and compared the behavior of these cells to vector transfected control cells. Expression of TFF3 was confirmed by RT-PCR for TFF3 mRNA (FIG. 1.). Expression of TFF3 increased mitogenesis of human mammary carcinoma cells (as indicated by 5′-bromo-2′-deoxyuridine labeling of nuclei) (FIG. 2A) and increased cell survival in serum deprived conditions (FIG. 2B). Consequently TFF3 produced an increase in total mammary carcinoma cell number in both serum deprived and serum containing conditions (FIG. 2C, D).

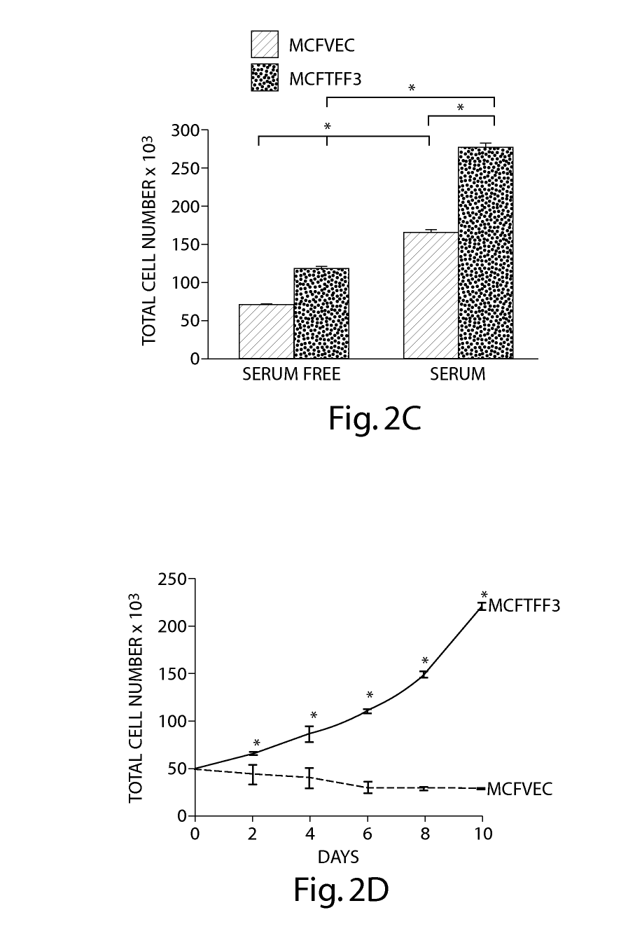

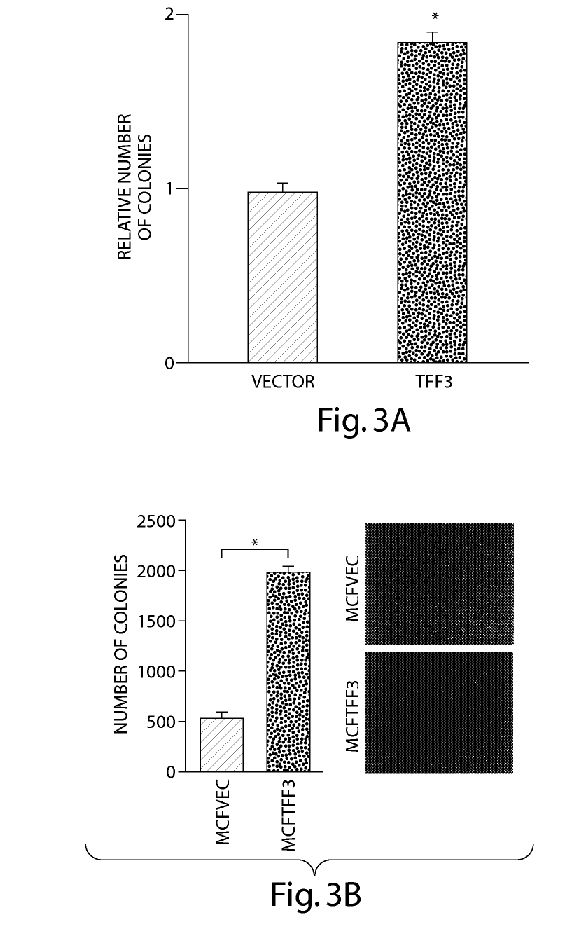

TFF3 increases anchorage-independent growth in human mammary carcinoma cells. One characteristic of oncogenically transformed cells is the capacity for anchorage independent growth. One measure of anchorage independent growth is the ability of transformed cells to form colonies in soft agar. To determine whether forced expression of TFF3 would regulate anchorage independent growth of human mammary carcinoma cells the inventors determined soft agar colony formation transient after forced expression of TFF3 in MCF-7 cells. Transient transfection of human mammary carcinoma cells with TFF3 cDNA increased the capacity for anchorage-independent growth compared to that of vector transfected cells as indicated by soft agar colony formation (FIG. 3A) MCF-7 cells with stable expression of TFF3 also produced dramatically more colonies in soft agar than vector transfected control cells (FIG. 3B). Another index of oncogenicity is proliferation in suspension culture and TFF3 production by mammary carcinoma cells similarly increases mammary carcinoma cell proliferation in suspension culture (FIG. 4A). Yet another index of oncogenecity is foci formation and TFF3 production by mammary carcinoma cells increases foci formation (FIG. 4B).

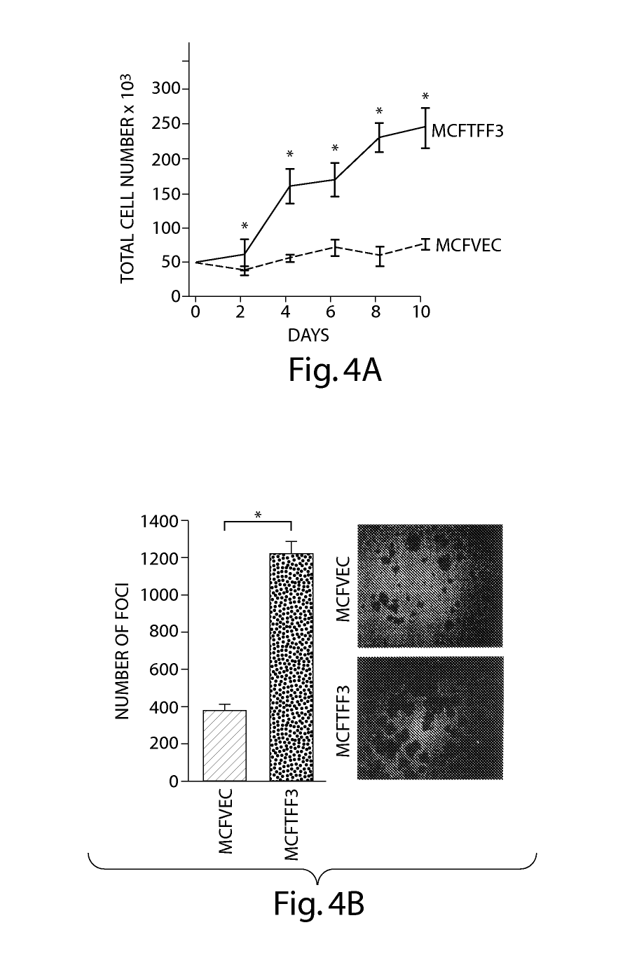

The human mammary carcinoma cell line used here also produces TFF3 endogenously. The inventors reasoned that if TFF3 was able to alter the capacity for anchorage independent growth, then knockdown of TFF3 would also abrogate the ability of mammary carcinoma cells to form colonies in soft agar. The inventors examined the anchorage independent growth of mammary carcinoma cells after transient transfection of a TFF3-RNAi construct generated as described in the materials and methods. siRNA to TFF3 reduced the level of TFF3 (FIG. 5A)). MCF-7 cells were transiently transfected either with the vector or TFF3-RNAi constructs and the soft agar colony formation was examined. Inhibition of TFF3 expression abrogated the number of colonies formed by MCF-7 cells in soft agar by more than half (FIG. 5B). An increase in TFF3 therefore increases oncogenicity of human mammary carcinoma cells and a decrease in TFF3 decreases oncogenicity of human mammary carcinoma cells. TFF3 therefore regulates the oncogenicity of human mammary carcinoma cells.

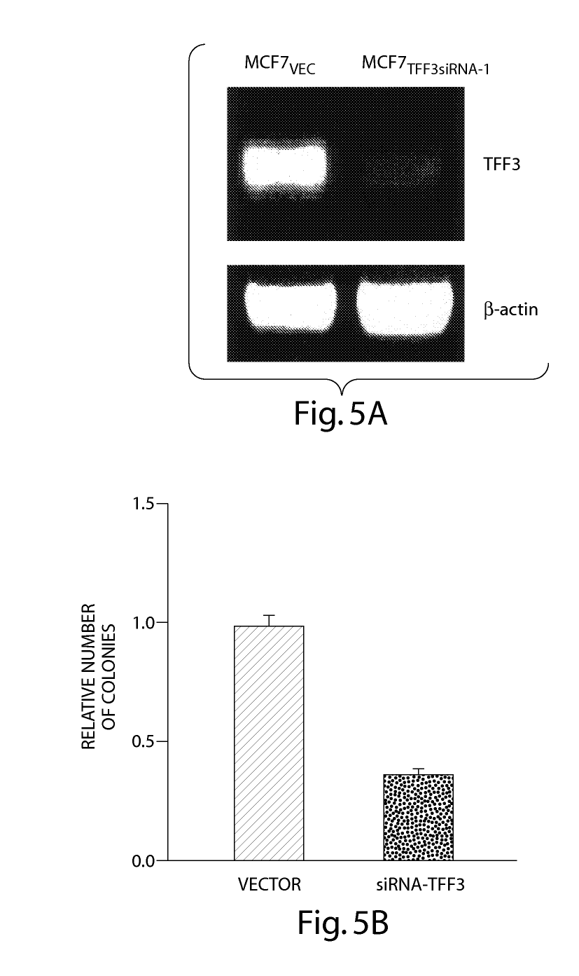

Forced expression of TFF3 in immortalized human mammary epithelial cells results in oncogenic transformation. To indicate if increased expression of TFF3 would result in oncogenic transformation of human mammary epithelial cells the inventors utilized the immortalized human mammary epithelial cell line MCF-10A. When grown attached to a plastic substrate these cells display normal epithelial morphology and do not form colonies in soft agar. To examine the oncogenic potential of TFF3 they transiently transfected MCF-10A cells with TFF3 cDNA or vector and examined for colony formation in soft agar. Control transfected cells were largely ineffective in colonization of soft agar whereas a significant number of colonies formed from cells transiently transfected with TFF3 (FIG. 6). This is remarkable given the relative inefficiency of transient transfection and that cells were grown for 14 days in soft agar before determination of colony formation. Thus increased expression of TFF3 is sufficient to support anchorage independent growth and potential to oncogenically transform immortalized human mammary epithelial cells.

These data indicate that that expression of TFF3 is sufficient to support anchorage independent survival and proliferation of human mammary epithelial cells and that TFF3 is a human mammary epithelial oncogene. The data also demonstrate the therapeutic benefits of inhibiting TFF3.

### Example 2

### Estradiol and Tamoxifen Treatment Induces Increase in Transcriptional Levels of TFF in Human Breast Adenocarcinoma Cells

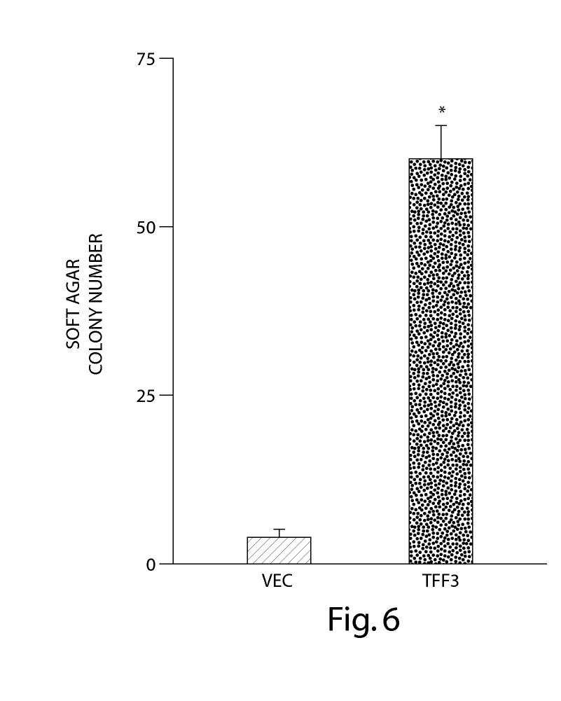

MCF7 cells have been shown to produce endogenous levels of TFF3, and TFF3 has been shown to be over-expressed in estrogen receptor positive breast cancers. It was of interest to determine if the expression of TFF3 is related to estrogen receptor status. MCF7 cells were cultured in phenol red free RPMI 1640 containing 10% dextran coated charcoal stripped fetal bovine serum in a 6 well plate for 24 hours. Then the cells were changed to treatment media containing 10 nM E2 or 1 μM TAM and cultured for 24 hours in the same. Untreated cells cultured in serum free media acted as controls. FIG. 7A shows very high increased transcript levels of TFF3 in cells treated with estradiol (E2) and significantly but not as high transcript levels of TFF3 when treated with tamoxifen (TAM). β-Actin was used for checking RNA integrity and also acted as a loading control. FIG. 7B shows an increase in TFF3 promoter activity after 24 hours treatment with E2 and TAM. *, p<0.05. These results show that E2 and TAM treatment induces increase in transcriptional levels of TFF3 in MCF7 cells.

### Example 3

### TFF3 Produces Resistance to Tamoxifen Induced Cell Death

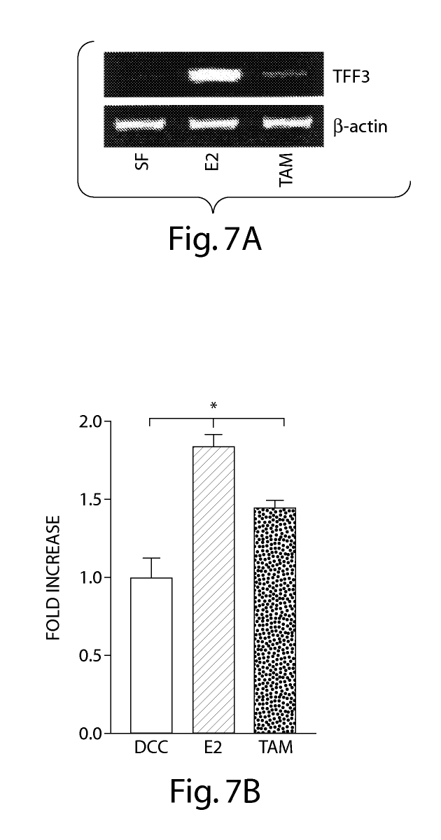

To determine if TFF3 would alter the responsiveness of human mammary carcinoma cells to agents used therapeutically to treat mammary carcinoma, the effect of forced expression of TFF3 was tested on soft agar colony formation in the presence of tamoxifen. MCF-7 cells with forced expression of TFF3 more than quadrupled soft agar colony formation in the presence of tamoxifen than vector transfected controls (FIG. 8). TFF3 therefore decreases the sensitivity of cells to the effects of antiestrogenic compounds used to treat mammary carcinoma.

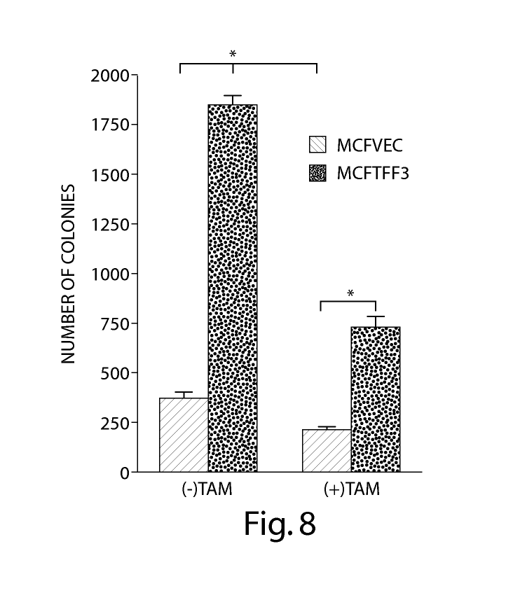

MCF7 cells produce endogenous levels of TFF1. MCF7 cells were cultured in phenol red free RPMI 1640 containing 10% dextran coated charcoal stripped fetal bovine serum in a 6 well plate for 24 hours. Then the cells were changed to treatment media containing 10 nM E2 or 1 μM TAM and cultured for 24 hours in the same. Untreated cells cultured in serum free media acted as controls. FIG. 9A shows very significant increase in transcript levels of TFF1 in cells treated with E2 and tamoxifen significantly. β-Actin was used for checking RNA integrity and also acted as a loading control. FIG. 9B shows an increase in TFF1 promoter activity after 24 hours treatment with E2 and TAM. *, p<0.001, **, p<0.0001. These results shows that estradiol and tamoxifen treatment induces an increase in transcriptional levels of TFF1 in MCF cells and that TFF1 and TFF3 are co-regulated and co-expressed in carcinoma of the breast.

### Example 4

### Estrogen Mediates MCF7 Cell Proliferation and Anchorage Independence Using TFF

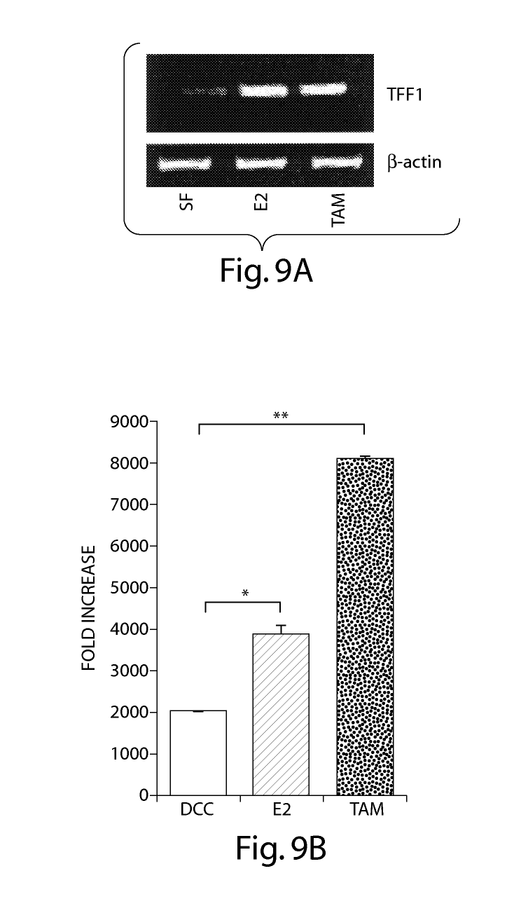

MCF7-VECTOR and MCF7-TFF3 cells were cultured in phenol red free RPMI 1640 media with 10% dextran coated charcoal stripped fetal bovine serum. Total cell number assay shows that removal of estrogen from the culture media reduces the total cell number and supplementing estrogen to the media brings back the total cell number to the control cells cultured in complete media (FIG. 10A). Soft agar colony formation assay shows, in estrogen depleted media, that TFF3 increases the total colony numbers by almost 10 fold and adding estrogen to the media further increases the colony numbers significantly in MCF-TFF3 (FIG. 10B). With estrogen treatment, the colony sizes were significantly larger in MCF-TFF3 cells compared to their controls. *, p<0.05 These results show that estrogen mediates cell proliferation and anchorage independence using TFF3.

### Example 5

### TFF is Overexpressed in Tamoxifen-Resistant Cells

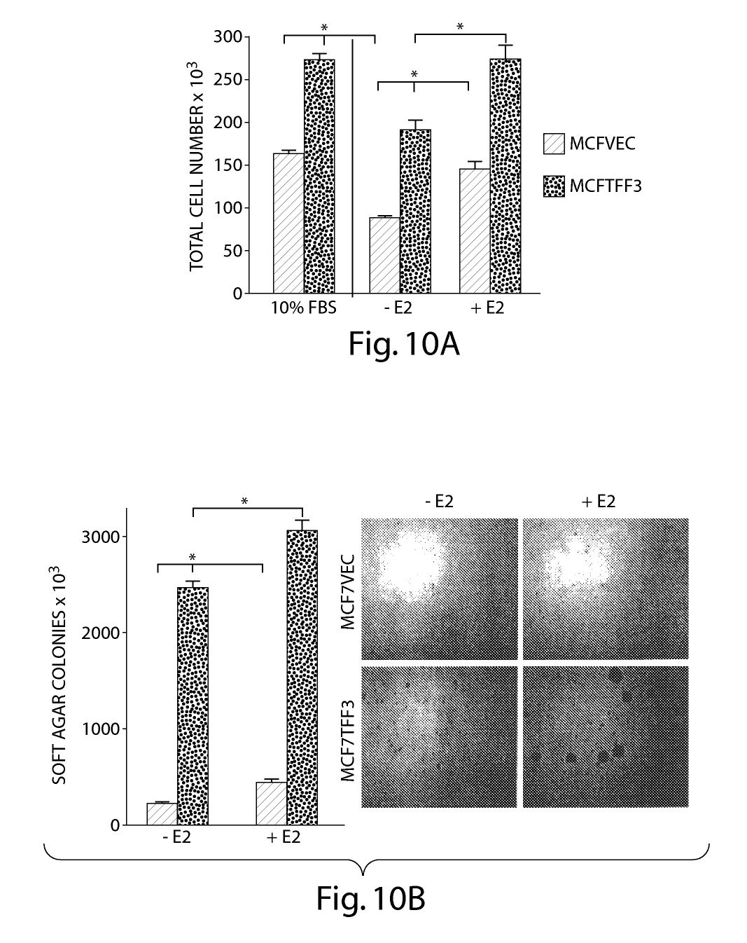

MCF7 cells were progressively exposed to increasing concentration of tamoxifen in phenol red free RPMI 1640 media with 10% dextran coated charcoal stripped fetal bovine serum. Cells were cultured in 1 μM for 6 months and tested for its resistance to tamoxifen compared to wild type MCF7 cells cultured in same media, which acted as a control, using MTT assay. The tamoxifen sensitive wild-type MCF7 (TAMS) cells and tamoxifen resistant (TAMR) cells were seeded equally in 6 well plates and transfected with TFF3-luciferase construct. Cultured in tamoxifen free media for 24 hours. Cells were lysed and using promega luciferase substrate kit, the enzyme activity was measured. TAMR shows increase more than 2-fold increase in TFF3 promoter activity compared to TAMS cells (FIG. 11A). FIG. 11B shows the TFF3 transcript levels in the cells tested and shows the upregulation of the transcript in the TAMR cells. β-Actin was used for checking RNA integrity and also acted as a loading control. *, p<0.05. These results show that TFF3 is over-expressed in tamoxifen-resistant (MCF-7) cells.

### Example 6

### Inhibition of TFF Production/Expression by Tumor Cells Using iRNA

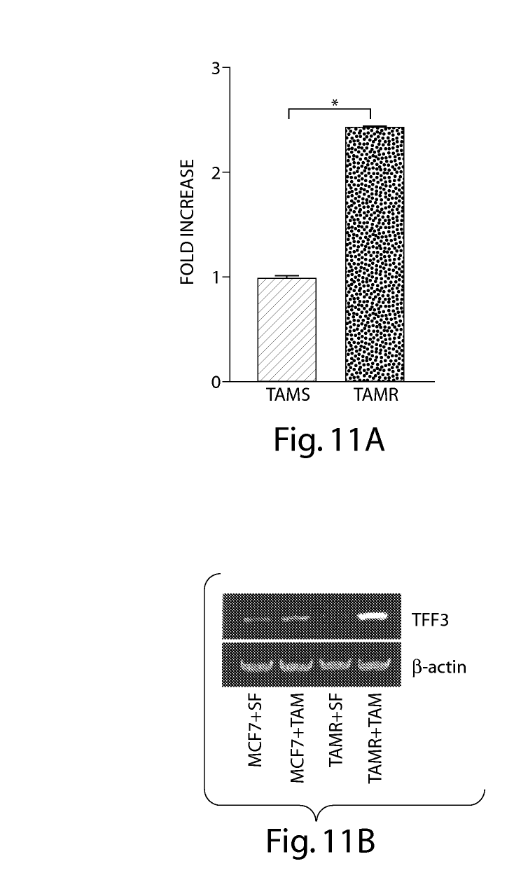

Experiments were carried out to determine the effect of TFF iRNA on tamoxifen sensitivity. TAMR and TAMS cells were transfected with pRNA U6.1-siRNATFF3. pRNA U6.1-VECTOR transfected cells acted as controls. 5000 cells from each group were embedded in soft agar. The cells were cultured with or without tamoxifen for two weeks. The colonies were scored as before. FIG. 12A shows that TFF3 mediates tamoxifen resistance in TAMR cells and TFF3 knock-down using siRNA increases tamoxifen sensitivity in these cells. *, p<0.05. These results show that TFF3 inhibition or knock-down using siRNA increases tamoxifen sensitivity in tamoxifen-resistant cells.

Further studies were carried out to determine the inhibitory effects of TFF1 and TFF3 siRNA, administered alone and in combination, on colony formation of MCF-7 cells on soft agar. MCF-7 cells were transiently transfected with 2 μg of TFF1 or TFF3 siRNA plasmids alone or combined together (1 μg each), or the empty siRNA vector as control by Saint Mix transfection reagent. 24 h post transfection, cells were trypsinised and 5,000 of each transfected cells were plated in six well plates in soft agar (0.35%) in RPMI 1640 containing 10% FBS. Colony formation was examined 12 later after crystal violet staining. TFF 1 siRNA and TFF3 siRNA combined together resulted in a significant reduction in the number of colonies in comparison to vehicle and even TFF1 or TFF3 siRNA alone (FIG. 12B).

### Example 7

### Inhibitory Effects of TFF Peptide Antagonists and TFF3-Human Serum Albumin Fusion Protein on TFF3 Activity

TFF1/TFF3 dimers are formed via intermolecular disulfide bonds between the C⁵⁷ amino acid residue, therefore replacement of this amino acid residue and addition of another protein such as human serum albumin or beta Casein will prevent TFF3 from forming dimers. The C57 to F point mutation of both TFF1 and TFF3, which is not able to form dimers as the wild type, inhibits the activity of endogenous TFF.

Further, in the TFF1/TFF3 dimer structure the amino acid residues Y²³, P²⁴, H²⁵, P⁴⁶ and W⁴⁷ form an exposed cleft between loops 2 and 3 on the protein surface consisting largely of hydrophobic residues, which is a binding site capable of accommodating either an oligosaccharide or a protein aromatic side chain and is important for its structure and homodimerization. Therefore, these residues are the targets for mutagenesis for the development of TFF1/3 antagonists. Antibodies that bind to these or adjacent residues inhibit TFF function such as oncogenicity and tumor cell proliferation and survival. Such antibodies inhibit TFF dimerization.

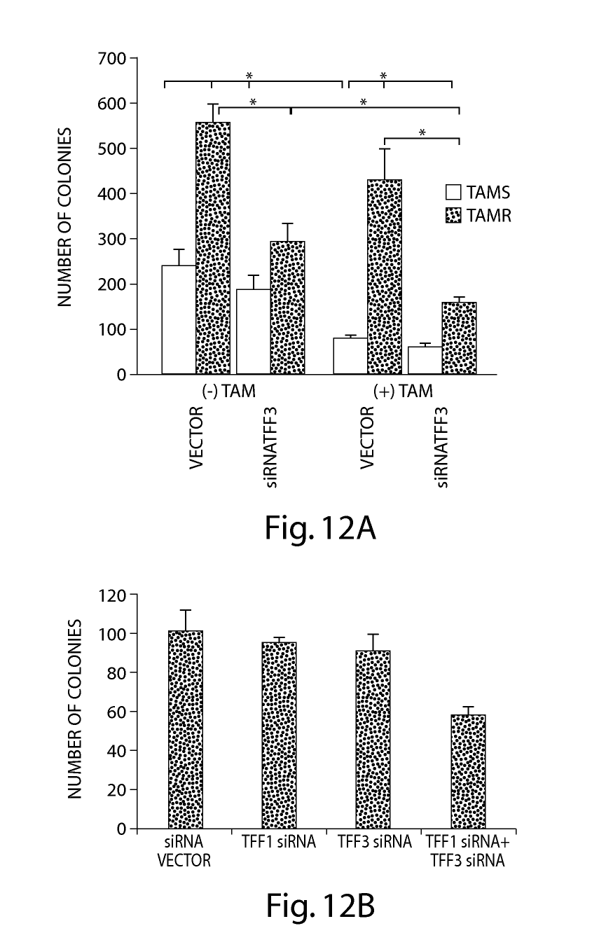

FIG. 13 shows the effects of TFF3 and its mutants on the transcription of CEACAM6 gene. MCF-7 cells were co-transfected with CEACAM6 luciferase report plasmid as well as expression plasmids of TFF3, c-Myc-Tagged (TFF3-tag), N-terminal deleted TFF3 (TFF3-Delta-1 and -2), TFF3-46R-47R and TFF3-C57F mutant, and TFF3-C57F-hSA (human serum albumin) fusion protein by Saint Mix transfection reagent as well as the empty vector as control. Luciferase activity was measured using a kit (Promega). Experiments were carried out in 6-well palates in three replicates.

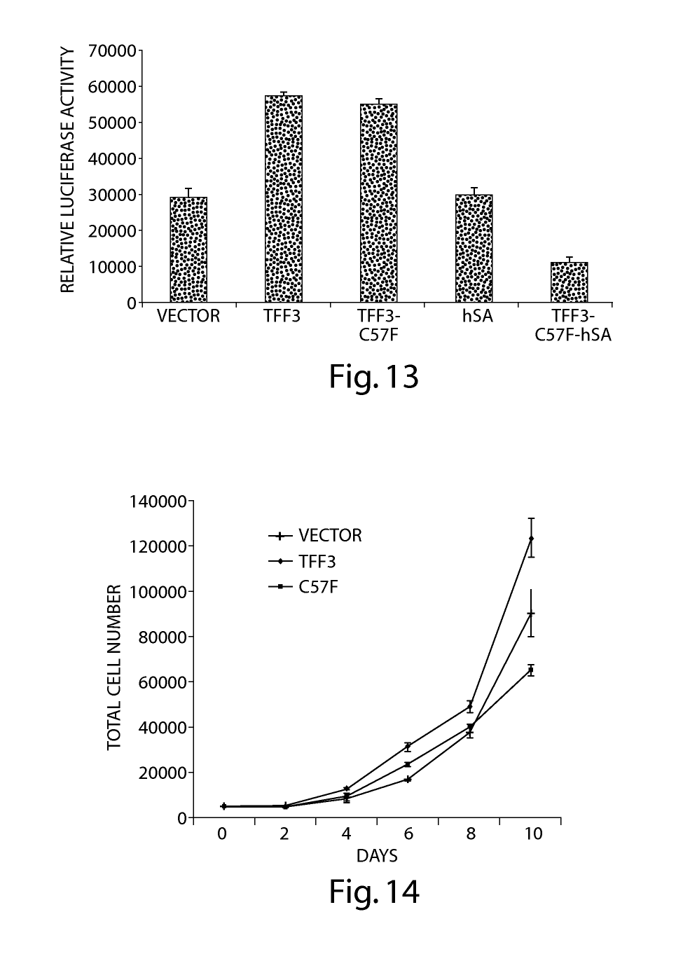

FIG. 14B shows the Growth curve of TFF3 and TFF3-C57F mutant stably expressing MCF-7 cells. Cells were plated in six well plates in RPMI 1640 containing 10% FBS. Total cell number were determined every 2 days.

TFF3 stimulated CECAM6 expression in MCF-7 cells by two fold compared with the control vector. While hSA (human serum albumin) or TFF3-C57F mutant alone had little effect on CEACAM6 expression, the TFF3-C57F-has fusion protein inhibited the expression of CECAM6 by 3 folds compared with the control vector. The cysteine at position of 57 of the mature TFF3 peptide is responsible to the formation of TFF3 dimers, however, removal of the cysteine did not appreciably affect the ability of TFF3 to activate CEACAM6 gene expression, indicating the monomer behaved the same way as dimer. However, when this mutant is fused to a large serum protein (65 kDa), the fusion protein worked as an antagonist to inhibit activity of wild type TFF3 on the activation of its downstream gene CEACAM6.

### Example 8

### Recombinant TFF Proteins and Anti-TFF Antibodies

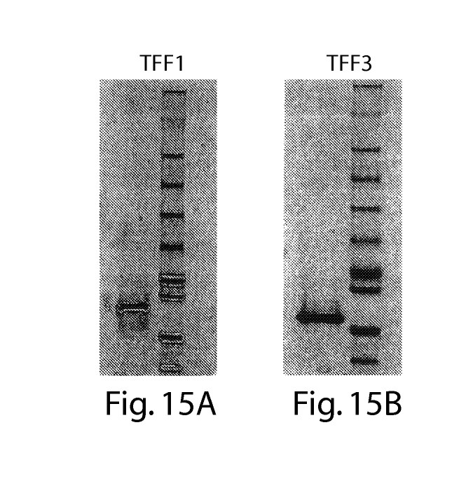

The cDNA coding for human TFF1 and TFF3 mature peptides were amplified using RT-PCR from RNA isolated from MCF-7 cells and cloned into the GST fusion protein expression vector pGEX 4T1 (FIG. 15). After transforming the pGEX4T1-TFF1 and pGEX4T1-TFF3 plasmids into BL21-Gold cells, the production of GST-TFF1 and GST-TFF3 were successfully achieved upon induction with IPTG. However, both GST-TFF1 and TFF3 fusion proteins were insoluble and thus could not be purified efficiently under a standard purification protocol. Therefore, a modified protocol was used to maximise the solubility of GST fusion proteins and subsequent binding to glutathione sepharose 4B matrix. TFF1 and TFF3 was cleaved from GST by on-column digestion with thrombin and eluted with PBS. The integrity and purity of the purified human TFF1 and TFF3 recombinant proteins were analysed by SDS-PAGE on a 4-12% NuPAGE Bis-tris gel and visualised by Coomassie Blue staining. TFF1 and TFF3 were shown as a 9 and 7 kD band, respectively, as expected.

Five milligrams of purified recombinant TFF1 and TFF3 proteins produced in E. coli were used to raise rabbit anti-TFF1 and TFF3 polyclonal antibodies. Two rabbits were immunized for each protein. The antibodies were affinity purified from the antisera. Thus, there are two polyclonal antibodies against each of TFF1 and TFF3 proteins: anti-TFF1-06 and anti-TFF1-07, and anti-TFF3-08 and anti-TFF3-09. Pre-immune sera were also taken for each as controls.

Antibodies produced bound to the native tertiary structure of TFFs in solution, i.e., the residues to which the antibodies bind are exposed on the surface of the tertiary structure of TFF1, 2, and 3 ((Williams et al., 2001, FEBS Lett. 493(2-3):70-74; Muskett et al., 2003, Biochemistry 42 (51):15139-15147, both of which are herein incorporated by reference).

### Example 9

### Anti-TFF Polyclonal Antibodies Inhibit MCF-7 Cell Proliferation in a Dose and Time Dependent Manner

MTT assay is based on that only metabolically active cells can cleave the yellow tetrazolium salt MTT to purple formazan crystals which are then solubilized and quantified by spectrophotometric means. Therefore, MTT assay has been accepted as one of the most sensitive and reliable cell biological approaches to quantitatively determine the cellular proliferation, viability and activation of a cell population's response to external factors. MCF-7 cells express both TFF1 and TFF3 at a relative high level. An MTT assay was used to test the effects of rabbit anti-TFF1 and TFF3 polyclonal antibodies on the proliferation of MCF-7 cells. MCF-7 cells were seeded into 96 wells microplates with affinity purified rabbit anti-TFF 1 or TFF3 antibodies at a concentration of 0, 1, 5 and 20 μg/ml of each antibody or their pre-immune sera as controls. To maintain the same concentrations, half of the indicated concentration for each antibody or serum was used when combined effects were studied. The cells were incubated for 48 h or 72 h and cell proliferation was determined by MTT assay using the procedure described in Material and Methods. CK-06, -07, -08 and -09 are the pre-immune sera for the rabbit polyclonal antibodies of human TFF1 (anti-F1-06 and -07) and TFF3 (anti-F3-08 and -09), respectively.

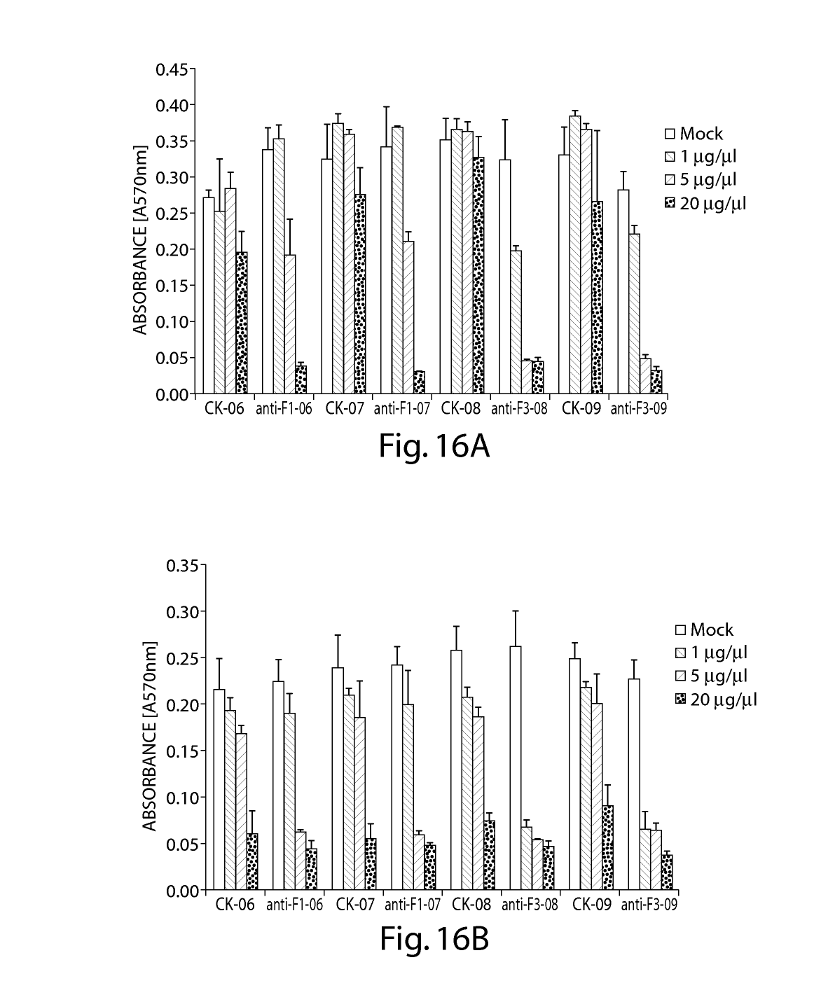

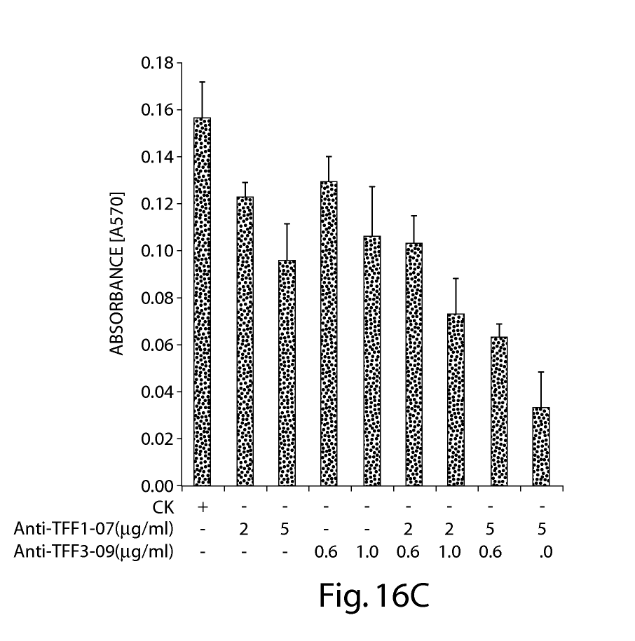

As shown in FIG. 16A, all the four polyclonal antibodies tested strongly inhibited MCF-7 cell proliferation in a dose dependent manner. After 48 h of incubation, the inhibitory effects of both anti-TFF1 and TFF3 antibodies on cell proliferation of MCF-7 cells were enhanced with increased antibody concentrations ranging from 0, 1, 5 up to 20 μg/ml. The inhibitory effects of both anti-TFF1 and -TFF3 antibodies on MCF-7 cell proliferation were not only dose dependent but also time dependent. As shown in FIG. 16B, after 72 h of incubation, 1 μg/ml of anti-TFF1 antibodies showed 15% inhibitory effects compared to the controls in the absent of antibodies, while the same concentration showed little inhibitory effects after an incubation period of 48 h. Again, after 72 h of incubation, 5 μg/ml of anti-TFF1 antibodies showed more than 70% inhibitory effects compared to the controls in the absent of antibodies, while the same concentration showed only 40% inhibitory effects after an incubation period of 48 h. The inhibitory effects of anti-TFF3 antibodies also became stronger with elongated time of incubation from 48 h to 72 h. Similarly, the cell proliferation were eventually blocked by anti-TFF3 antibodies at a concentration of 1 μg/ml after 72 h compared with the controls, while the cell proliferation was only reduced to 60% at the same concentration after 48 h.

The combined effects of anti-TFF1 and -TFF3 antibodies was also tested. Suboptimal concentrations of anti-TFF1 and TFF3 were more effective in combination than each TFF alone in MCF-7 cells (FIG. 16C). These data indicate that a combination of TFF-specific antibodies (e.g., anti-TFF1 and anti-TFF3, anti-TFF1 and anti-TFF2, or anti-TFF2 and anti-TFF3) produce a synergistic effect.

In all cases, pre-immune sera had no or little effects on MCF-7 cell proliferation except the 20 μg/ml treatments were shown to be cytotoxic to the cells after an incubation period of 72 h. Therefore, the inhibitory effects of anti-TFF1 and TFF3 antibodies were specific.

### Example 10

### Rabbit anti-TFF1 and TFF3 Polyclonal Antibodies Inhibited Anchorage-Independent Growth of MCF-7 Cells in Soft Agar

Like all other cancer cells, human mammary carcinoma MCF-7 cells have the capacity to form colonies in soft agar. 5×10³ MCF-7 cells were plated in six well plates in soft agar (0.35%) in RPMI 1640 containing 10% FBS with or without 5 μg/ml of rabbit anti human TFF1 or TFF3 antibodies and the preimmune sera as control. Colony formation was examined 10 later after crystal violet staining. Photos were taken at 10× magnification.

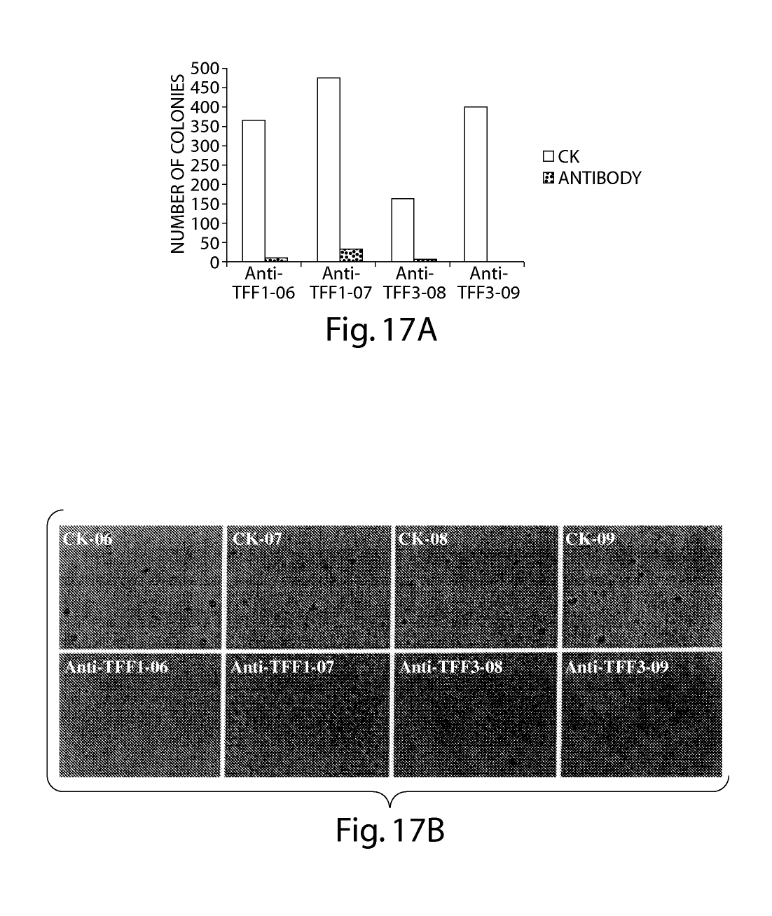

As shown in FIGS. 17A and 17B, inclusion of either anti-TFF1 or anti-TFF3 polyclonal antibodies remarkably reduced the colony formation in soft agar. In the presence of pre-immune sera, the number of colonies formed varied from 160 to 400 among different treatments, whereas there were only 10, 35, 8 and 1 colonies formed in the presence of anti-TFF1 antibodies (anti-TFF1-06 and -07) and anti-TFF1 antibodies (anti-TFF1-08 and -09), respectively. Therefore, the rabbit anti-TFF1 and TFF3 polyclonal antibodies not only prevented cell proliferation of MCF-7 cells as shown in the MTT assays, but also their anchorage-independent growth in soft agar. These data indicate that TFF-specific antibodies reduce oncogenicity of endogenous TFF.

### Example 11

### Anti-TFF Polyclonal Antibodies Inhibit Cell Proliferation of Tamoxifen-Resistant MCF-7-Tam^{R} Cells

MCF-7 cells are estrogen-receptor (ER) positive and anti-estrogen drug tamoxifen blocks the proliferation of the cells. MCF-7-Tam^{R} cells are a derivative of original MCF-7 cells and were established by progressively exposing to increasing concentrations of tamoxifen. Cells were plated in phenol red free RPMI 1640 medium containing dextran coated charcoal stripped 10% FBS at 5×10³ cells/100 μl/well in 96-well plates. After 24 hours' incubation, anti-TFF3 antibody and/or tamoxifen (TAM) were then added at twice the final concentration in a volume of 100 μl. Cell proliferation was determined by MTT assay. CK-09 is the pre-immune sera for the rabbit polyclonal antibodies of human TFF3 (anti-09).

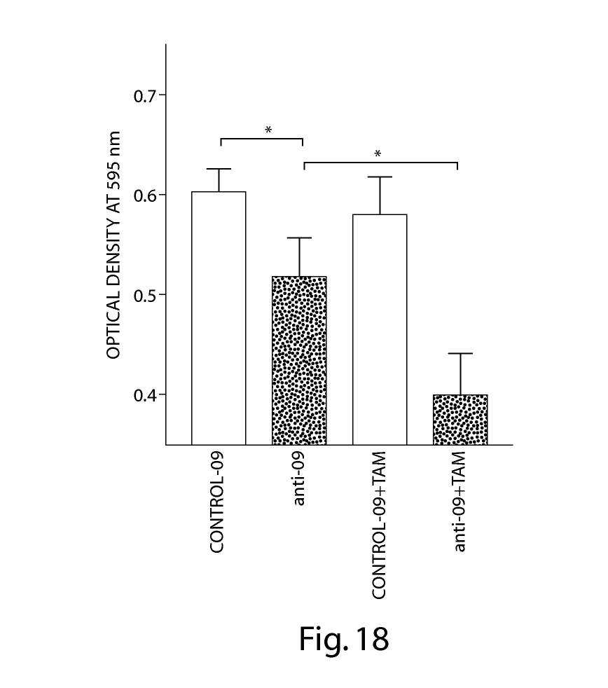

As shown in FIG. 18, MCF-7-Tam^{R} cells were sensitive to anti-TFF3 antibody treatment. In the absence of tamoxifen, 5 μg/ml of anti-TFF3 antibody decreased cell proliferation by 30% compared with control and by 20% compared with the pre-immune serum. In the presence of 1 μM of tamoxifen, anti-TFF3 antibody caused significant cell death compared to the pre-immune serum. Therefore, tamoxifen enhanced the inhibitory effect of anti-TFF3 antibody on cell proliferation of tamoxifen-resistant MCF-7-TamR cells.

### Example 12

### Morphological Changes of Tamoxifen-Resistant MCF-7-Tam^{R} Cells by Rabbit Anti-TFF3 Polyclonal Antibody

Cells were plated in phenol red free RPMI 1640 medium containing dextran coated charcoal stripped 10% FBS at 5×10³ cells/100 μl/well in 96-well plates. After 24 hours incubation, anti-TFF3 antibody and/or tamoxifen (TAM) were then added at twice the final concentration in a volume of 100 μl. The cells were then taken photos using Olympus phase contrast microscope under 10× magnification. CK-09 is the pre-immune sera for the rabbit polyclonal antibodies of human TFF3 (anti-09).

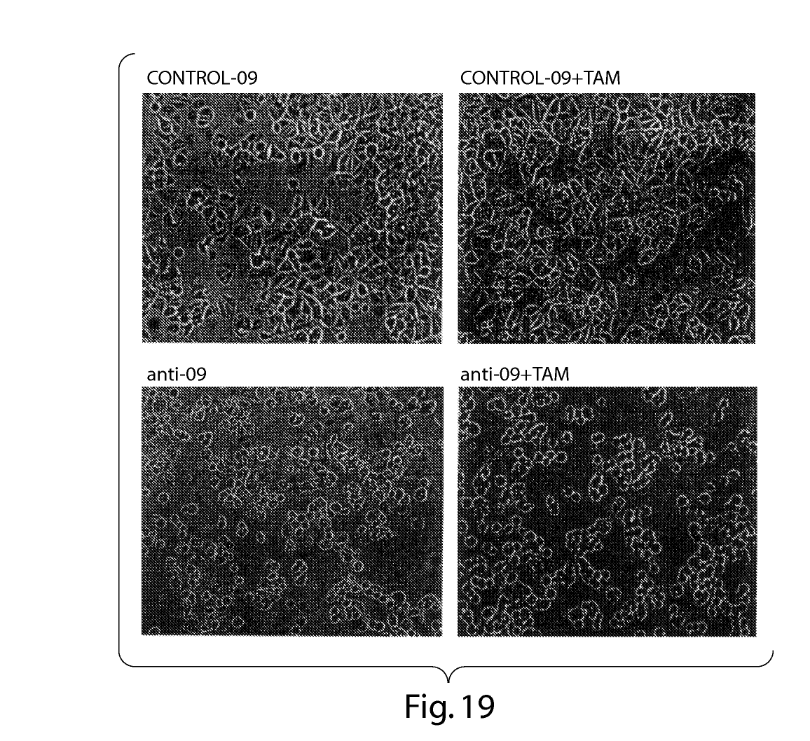

As shown in FIG. 19, the cell morphological assay confirms the MTT data and clearly shows that by blocking/neutralizing TFF3 peptide using polyclonal anti-TFF3 antibody, the MCF-7-Tam^{R} cells die. MCF-7-Tam^{R} cells were cultured in 1 μM TAM for more than 9 months and were shown to be completely resistant to TAM treatment. Under experimental conditions, TAM alone treated cells look healthy and similar to untreated control showing resistance to TAM induced cell death. Consistent with soft agar colony formation assay (FIG. 17) in which the antibody treatment killed most of the wild type MCF-7 cells, we here found that MCF-7-Tam^{R} cells when treated with antibody alone, showed very different morphology compared to the controls, revealing that the treatment resulted in cell death. The cells looked shrunken and rounded but still some flattened cells growing normally were seen. MCF-7-Tam^{R} cells when treated with both anti-TFF3 antibody and TAM caused significant death.

### Administration of Compositions for Cancer Therapy

The TFF-specific antibodies described herein are used to increase tamoxifen sensitivity, increase sensitivity to hormone-based therapy (e.g., anti-estrogens) inhibit the growth of a tumor cell, or kill the tumor cell. In addition to cancer therapy, the methods are useful to confer clinical benefit to those suffering from or at risk of developing a precancerous condition or lesion or a non-cancerous hyperproliferative disorder.

Purified antibody preparations (e.g., a purified polyclonal or monoclonal antibody, an antibody fragment, or single chain antibody) is administered to an individual diagnosed with a tumor or at risk of developing a tumor that expresses an increased level of TFF (compared to a normal cell of the same tissue type). The antibody preparations are administered using methods known in the art of passive immunization, e.g., intravenously or intramuscularly. Alternatively, the preparation is administered locally to a tumor site. The antibodies used in the methods described herein are formulated in a physiologically-acceptable excipient. Such excipients, e.g., physiological saline, are known in the art. Optionally, tamoxifen or another chemotherapeutic drug is administered in a combination therapy approach. The antibody is preferably a high-affinity antibody, e.g., an IgG-class antibody or fragment or single chain thereof. Antibodies are optionally humanized.

Peptide antagonists and antibody preparations (or antibody-toxin preparations) are administered at a standard dosing schedule of 375 mg/m² weekly. An alternate standard dosing regimen is 4 mg/kg administered as a 90-minute infusion with a weekly maintenance dose is 2 mg/kg administered as a 30-minute infusion. Such regimens are known in the art. The dose is altered depending on co-administered compositions (e.g., tamoxifen or other drug) and depending on the response of the patient. Doses are readministered weekly or monthly as necessary to reduce tumor load in a treated individual.

Nucleic acid constructs (antisense, siRNA, protein-, peptide-encoding constructs) are administered systemically or locally using known methods. An DNA or mRNA dosage is generally be in the range of from about 0.05 micrograms/kg to about 50 mg/kg, usually about 0.005-5 mg/kg of body weight, e.g., 0.5 to 5 mg/kg. Dosage for intravenous administration of nucleic acids is from approximately 10⁶ to 10²² copies of the nucleic acid molecule.

Thus, methods of regulating cellular proliferation and/or differentiation involve at least the step of inhibiting one or more TFF proteins. The methods are practiced in a cell (in vitro) or in a subject (in vivo). Preferably, one or more of TFF1, TFF2, or TFF3 proteins are inhibited. If more than one TFF product e.g., TFF1 and TFF3, TFF1 and TFF2, or TFF2 and TFF3, is inhibited, the effect is synergistic, i.e. a suboptimal dose of each inhibitory compound given together achieves a beneficial therapeutic effect compared to the amount required for one inhibitor administered alone. For example, the amount of total antibody with a TFF1/3 mixture required for a similar effect is less than that required with either TFF1-specific antibody or TFF3-specific antibody alone, e.g., at least 10, 20, 30, 40, 50% less of each TFF antibody is used in combination with another TFF antibody to achieve a clinical benefit.

Inhibitory compounds include a nucleic acid adapted to inhibit TFF or a peptide antagonist of TFF. The nucleic acid is an antisense nucleic acid to a TFF transcript, or a nucleic acid adapted to express such an antisense; iRNA to a TFF transcript, or a nucleic acid adapted to express such iRNA. Preferably, the nucleic acid inhibits TFF1, TFF2 or TFF3 transcription or translation.

The present invention also provides compositions comprising one or more nucleic acid adapted to inhibit a TFF in use or a peptide antagonist of TFF together with one or more pharmaceutically acceptable carriers, diluents and/or excipients.

“Proliferative disorders” are those disorders resulting from aberrant proliferation of one or more cell type within a subject. Such disorders may be benign or malignant. The disorder can be cancer or a hyperproliferative disorder. Various cancers to be treated include but are not limited to lung cancer, colorectal cancer, breast cancer, pancreatic cancer, ovarian cancer, prostate cancer, renal carcinoma, hepatoma, brain cancer, melanoma, multiple myeloma, hematologic tumor, and lymphoid tumor. Preferably the cancer is breast cancer and more preferably the cancer is tamoxifen-resistant. Various hyperproliferative disorders, to be treated include but are not limited to keratinocyte hyperproliferation, inflammatory cell infiltration, cytokine alteration, epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses.

As used herein, the term “subject” includes any animal of interest. In particular the invention is applicable to mammals, more particularly humans. The term “treatment” includes the modulation or control of a proliferative disorder, amelioration of the symptoms or severity of a particular disorder, or preventing or otherwise reducing the risk of developing a particular disorder. The term does not necessarily imply that a subject is treated until total recovery.

“Inhibition” of a TFF protein is intended to refer to blocking, lowering or reducing the production biological activity of the protein. While it may be desirable to completely inhibit the activity of a TFF protein, a 5, 10, 20, 25, 50, 75, 90 and up to 100% inhibition compared to a pre-treatment level of TFF protein or activity confers a therapeutic benefit. “Inhibition” of a TFF protein may occur at the level of expression and production of a TFF protein (for example the transcriptional or translational level) or by targeting the function of a TFF protein.

The invention relates to various means and agents of use to inhibit a TFF protein. By way of example, nucleic acid technology including iRNA, antisense and triple helix DNA may be employed to block expression. Further examples include the use of specific antagonists of TFF proteins, including peptide antagonists, and antibodies directed against TFF proteins, or functional derivatives of such antibodies. Antibodies and derivatives thereof include for example, intact monoclonal antibodies, polyclonal antibodies, hybrid and recombinant antibodies (including humanised antibodies, diabodies, and single chain antibodies, for example), and antibody fragments so long as they exhibit the desired activity.

The efficacy or therapeutic benefit of an agent in inhibiting a TFF is determined by detecting a reduction in tumor load or tumor mass. Efficacy of agents is also determined by detecting of mitogenesis, cell survival, cell numbers, proliferation. Preferred TFF inhibitors one or more of the following characteristics: 1) the ability to prevent, decrease or inhibit mitogenesis; 2) the ability to prevent, decrease or inhibit cell survival; 3) the ability to prevent or inhibit the increase in cell numbers or to decrease cell numbers; 4) the ability to prevent or abrogate anchorage independent growth or encourage or maintain anchorage dependent growth; and, 5) the ability to prevent, inhibit or decrease oncogenic transformation. Preferably suitable agents will exhibit two or more of these characteristics.

Nucleic acids are utilized to inhibit a TFF protein. Such nucleic acids may be DNA, RNA, single-stranded, or double-stranded. Nucleic acids of use in the invention may be referred to herein as ‘isolated’ nucleic acids. “Isolated” nucleic acids are nucleic acids which have been identified and separated from at least one contaminant nucleic acid molecule with which it is associated in its natural state. Accordingly, it will be understood that isolated nucleic acids are in a form which differs from the form or setting in which they are found in nature. It will further be appreciated that ‘isolated’ does not reflect the extent to which the nucleic acid molecule has been purified.

Isolated nucleic acids of used in the invention may be obtained using a number of techniques known in the art. For example, recombinant DNA technology may be used as described for example in Joseph Sambrook and David W. Russell. Molecular Cloning: A Laboratory Manual (Third Edition), Cold Spring Harbor Laboratory Press, New York, USA. Similarly chemical synthesis (for example, using phosphoramidite and solid phase chemistry) may be used.

Nucleic acids of use in the invention may be designed on the basis of particular TFF nucleic acid sequence data, the known relative interactions between nucleotide bases, and the particular nucleic acid technology to be employed, as may be exemplified herein after. Sequence similarities exist between TFF proteins and genes within species and between species, that a nucleic acid designed against one TFF gene/transcript may be of use in inhibition of a related TFF, e.g., the invention provides nucleic acids designed around TFF1 are useful in inhibiting TFF3.

Interference RNA (iRNA) or short interfering RNA (siRNA) are utilized to inhibit a TFF. The iRNA and siRNA are used interchangeably herein. Nucleic acids of use in iRNA techniques will typically have 100% complementarity to their target. However, it should be appreciated that this need not be the case, provided the iRNA retains specificity for its target and the ability to block translation. Exemplary iRNA molecules may be in the form of ˜18 to 21 by double stranded RNAs with 3′ dinucleotide overhangs, although shorter or longer molecules may be appropriate. In cases where the mRNA is produced in vivo by an appropriate nucleic acid vector, it will typically take the form of an RNA molecule having a stem-loop structure (for example having an approximately 19 nucleotide stem and a 9 nucleotide loop with 2-3 Us at the 3′ end). Algorithms of use in designing siRNA are available from Cenix (Dresden, Germany—via Ambion, Texas USA).

The present invention provides one or more iRNA molecules to a TFF1 transcript or a nucleic acid adapted in use to express such mRNA. An iRNA can be chosen from the group targeting the following sequences:

| | |
|---|---|
| 5′-AATGGCCACCATGGAGAACAA-3′ | (SEQ ID NO: 1) |
| 5′-AAATAAGGGCTGCTGTTTCGA-3′ | (SEQ ID NO: 2) |

An mRNA to TFF1 can be chosen from the group having the following structures:

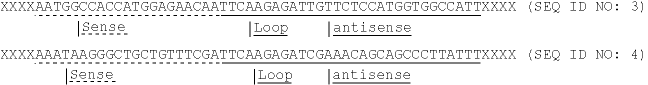

Exemplary human nucleic acid and amino acid sequence data for TFF1 is provided on GenBank under the accession number NM_{—}003225, herein incorporated by reference. Orthologues have also been described in other primates, and in rat and mouse. Exemplary rat sequence data is provided on GenBank under the accession number NM_{—}057129, herein incorporated by reference. Exemplary murine sequence data is provided under the accession number NM_{—}009362, herein incorporated by reference.

The present invention also provides one or more iRNA molecules to a TFF2 transcript or a nucleic acid adapted in use to express such iRNA. An iRNA can be chosen from the group targeting the following sequences:

| | |
|---|---|
| 5′-AATCACCAGTGACCAGTGTTT-3′ | (SEQ ID NO: 5) |
| 5′-AATGGATGCTGTTTCGACTCC-3′ | (SEQ ID NO: 6) |

An iRNA to TFF2 can be chosen from the group having the following structures:

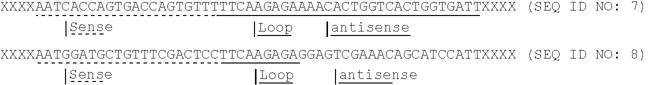

Exemplary human nucleic acid and amino acid sequence data for TFF2 is provided on GenBank under the accession number NM_{—}005423, herein incorporated by reference. Orthologues in rat and mouse have been described. Exemplary rat sequence data is provided under the accession number NM_{—}053844, herein incorporated by reference. Exemplary mouse data is provided under the accession number NM_{—}009363, herein incorporated by reference.

The present invention also provides one or more iRNA molecules to a TFF3 transcript or a nucleic acid adapted in use to express such mRNA. In one aspect, an iRNA is chosen from the group targeting the following sequences:

| | |
|---|---|
| 5′-ACTAGGAAGACAGAATGCA-3′ | (SEQ ID NO: 9) |
| 5′-ACTAGGAAGACAGAATGC-3′ | (SEQ ID NO: 10) |
| 5′-TGCTTTGACTCCAGGATCC-3′ | (SEQ ID NO: 11) |

An iRNA to a TFF3 transcript are chosen from the group having the following structures:

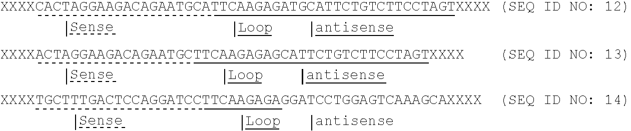

Exemplary human nucleic acid and amino acid sequence data for TFF3 is provided on GenBank under the accession number NM_{—}003226, herein incorporated by reference. Orthologues in other primate species and in rat and mouse have been described. Exemplary rat TFF3 information is found on GenBank under the accession number NM_{—}013042, herein incorporated by reference. Exemplary murine sequences are found under the accession number NM_{—}011575, herein incorporated by reference. Exemplary primate data is provided under the accession numbers XM_{—}525480 and XP_{—}525480, herein incorporated by reference. The structure of TFF3 has been elucidated by Muskett et al (Biochemistry 42(51):15139-47, 2003).

XXXX indicates additional nucleotides which may be present; for example termination signals and restriction sites which may be of use in cloning and expressing the iRNA. By way of example, the following nucleic acids may be used to clone and express (in desired vectors) iRNAs:

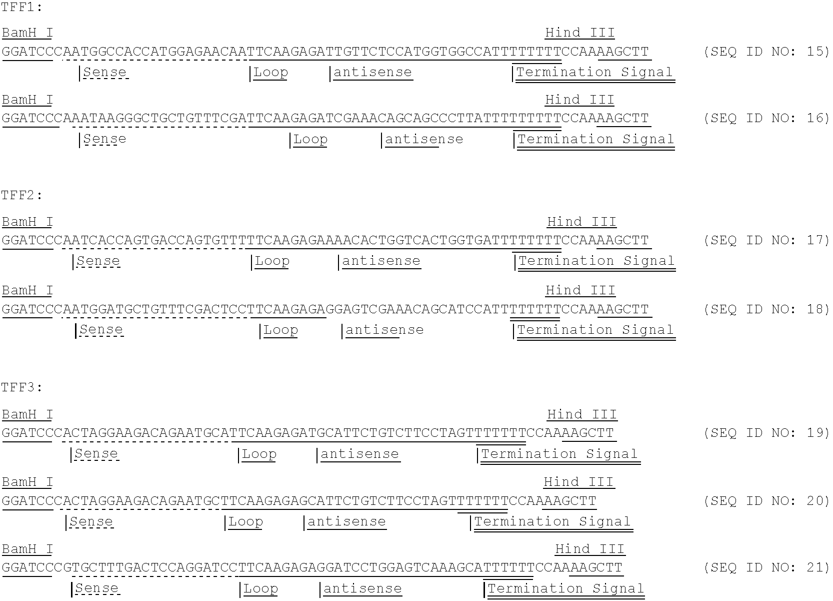

iRNA molecules are produced in accordance with techniques described herein and those known in the art. Further information regarding how to produce and design such molecules can be gained, for example, from: McManus and Sharp, Nature Rev Genet 3: 737-747, 2000; Dillin, Proc Natl Acad Sci USA 100(11): 6289-6291, 2003; and Tuschl, Nature Biotechnol 20: 446-448, 2002.

Antisense molecules to inhibit TFF production by a tumor cell. Antisense means any nucleic acid (preferably RNA, but including single stranded DNA) that binds to a TFF transcript to prevent translation thereof. Typically, antisense molecules or oligonucleotides consist of 15-25 nucleotides which are completely complementary to their target mRNA. However, larger antisense oligonucleotides including full-length cDNAs are also inhibitory. Antisense molecules which are not completely complementary to their targets are utilized provided they retain specificity for their target and the ability to block translation.

Nucleic acid molecules of use in the invention, including antisense, iRNA, ribozymes and DNAzymes may be chemically modified to increase stability or prevent degradation or otherwise. For example, the nucleic acid molecules may include analogs with unnatural bases, modified sugars (especially at the 2′ position of the ribose) or altered phosphate backbones. Such molecules may also include sequences which direct targeted degradation of any transcript to which they bind. For example, a sequence specific for RNase H, may be included. Another example is the use of External Guide Sequences (EGSs), which may recruit a ribozyme (RNase P) to digest the transcript to which an antisense molecule is bound for example.

Inhibitory nucleic acids are in the form of synthetic nucleic acid molecules produced in vitro (for example single stranded DNA, iRNA, antisense RNA, DNAzymes), or alternatively, they are encoded by sequences in a vector to produce an active inhibitory compound, e.g., antisense molecules, IRNA, ribozymes. Any suitable vector known in the art is within the scope of the present invention. For example, naked plasmids that employ CMV promoters are used. Standard viral vectors such as adeno-associated virus (AAV) and lentiviruses are suitable. Such vectors are known in the art: the use of retroviral vectors is reported in Miller et al., Meth. Enzymol. 217:581-599, 1993 and Boesen et al., Biotherapy 6:291-302, 1994; the use of adenoviral vectors is reported for example in Kozarsky and Wilson, Current Opinion in Genetics and Development 3:499-503, 1993; Rosenfeld et al., Science 252:431-434, 1991; Rosenfeld et al., Cell 68:143-155, 1992; Mastrangeli et al., J. Clin. Invest. 91:225-234, 1993; PCT Publication WO 94/12649; and Wang, et al., Gene Therapy 2:775-783, 1995; and, the use of AAV has been reported in Walsh et al., Proc. Soc. Exp. Biol. Med. 204:289-300, 1993; U.S. Pat. No. 5,436,146. Other examples of suitable promoters and viral vectors are provided herein.

Nucleic acid vectors or constructs of use in the invention may include appropriate genetic elements, such as promoters, enhancers, origins of replication as are known in the art, including inducible, constitutive, or tissue-specific promoters. A vector can comprise an inducible promoter operably linked to the region coding a nucleic acid of the invention (for example antisense TFF3 or suitable siRNA), such that expression of the nucleic acid is controllable by controlling the presence or absence of the appropriate inducer of transcription. Nucleic acid molecules encoding a peptide of the invention can be flanked by regions that promote homologous recombination at a desired site in the genome, thus providing for intrachromosomal integration of the desired nucleic acids (Koller and Smithies, 1989, Proc. Natl. Acad. Sci. USA 86:8932-8935; Zijlstra et al., 1989, Nature 342:435-438). Of course, the vectors may remain extrachromosomal.

Peptides and/or proteins may be utilized to inhibit a TFF protein in accordance with the invention. A “peptide antagonist” is a peptide having the ability in use to block, lower or reduce biological activity of a TFF. While it may be desirable to completely inhibit the activity of a TFF, this need not be essential. Peptide antagonists include those peptides that compete with native TFF for binding to a TFF receptor, prevent native TFF binding to a TFF receptor, prevent dimerization of TFF or a TFF receptor, or prevent activation of a TFF receptor.

A peptide or protein is an “isolated” or “purified” peptide. An “isolated” or “purified” peptide is one which has been identified and separated from the environment in which it naturally resides. It should be appreciated that ‘isolated’ does not reflect the extent to which the peptide has been purified or separated from the environment in which it naturally resides. Preferably, the peptide of interest is at least 60%, by weight, of the protein in the preparation. Preferably, the protein in the preparation is at least 75%, more preferably at least 90%, and most preferably at least 99%, by weight. Purity is measured by any appropriate method, e.g., column chromatography, polyacrylamide gel electrophoresis, or HPLC analysis.

Peptide antagonists are designed on the basis of the published amino acid and nucleic acid sequence data in respect of a TFF as described herein. For instance, a peptide antagonist are derived from a native TFF amino acid sequence incorporating one or more mutation therein. Such mutations include for example amino acid insertions, deletions, substitutions and the like. Alternatively, a peptide antagonist is a fragment of the full length native TFF protein, which may or may not include a mutation(s). Peptide antagonists may also include fragments of the native TFF protein fused to a heterologous peptide. For example, the heterologous peptide (e.g., human serum albumin) serves to increase serum half-life (i.e., decrease protein degradation) and/or decrease rapid excretion of the construct by the kidneys. The heterologous peptide may also serve a mass effect of preventing or impairing interaction of TFF with its receptor or receptor activation.

### Inhibition of TFF3

Several amino acid residues (i.e., Tyr 23, Pro 24, His 25, Pro 46, and Trp 47) of TFF3 are important for the structure and potential function of TFF3. Further, amino acid residue, Cys 57, is of importance in forming intermolecular disulfide bonds. Accordingly, peptide antagonists include an alteration at one or more of the above sites based of the native TFF3 amino acid sequence. For example, hydrophobic amino acids Tyr 23, Pro 24, or Trp 47 are replaced by Arg or Lys. Further examples include peptides in which Cys 57 is replaced by Phe. Alternatively, Cys 57 is followed by human beta casein or another peptide sequence; i.e., the TFF3 protein is truncated and fused to a heterologous peptide or protein.

As used herein, the numbering associated with the amino acid positions of TFF3 are as based on the following, wherein number starts from the amino acid marked *1:

| M Q E R T G A A T A R R E S L P Q A N N P E Q L C K Q R C I N E A | (SEQ ID NO: 22) |
|---|---|
| S W T M K R V L S C V P E P T V V M A A R A L C M L G L V L A L L | |
| S S S S A E E Y V G L S A N Q C A V P A K D R V D C G Y P H V T P | |
| *1 28 | |
| K E C N N R G C C F D S R I P G V P W C F K P L Q E A E C T F | |
| 29 59 | |

The amino acid sequence MQERTGAATARRESLPQANNPEQLCKQRCINEAS WTMKRVLSCVPEPTVV (SEQ ID NO:23) represents a TFF3 precursor start sequence. The amino acid sequence MAARALCMLGLVLALLSSSSA (SEQ ID NO:24) represents a TFF3 signal peptide sequence. The amino acid sequence EEYVGLSANQCAVPAKDRV DCGYPHVTPKECNNRGCCFDSRIPGVPWCFKPLQEAECTF (SEQ ID NO:25) represents the mature form of the TFF3 polypeptide. TFF inhibitory activity antagonists is determined by observing their ability to bind to TFF3, compete with TFF3 for receptor binding, prevent TFF3 or receptor dimerisation, prevent activation of a TFF receptor or generally inhibit the functional effects of TFF3 in cells.

TFF1 and TFF2 mutants that function as TFF antagonists were made by replacing the analogous residues in the structure of the TFF1 and TFF2 sequences, respectively as shown in the alignment below.

| | | | | |
|---|---|---|---|---|
| hTFF3 | 1 | MQERTGAATARRESLPQANNPEQLCKQRCINEASWTMKRVLSCVPEPTVVMAARALCMLG | 60 | (SEQ ID NO: 22) |
| hTFF1 | 1 | -----------------------------------------------MATMENKVICALV | 13 | (SEQ ID NO: 33) |
| hTFF2a | 1 | -----------------------------------------------MGRRDAQLLAAL- | 12 | (SEQ ID NO: 34) |
| hTFF2b | 0 | ------------------------------------------------------------ | 0 | (SEQ ID NO: 35) |
| | | \|mature peptide RR RR | | |
| hTFF3 | 61 | LVLAL-LSSSSA\|EEYVGLSANQC-AVPAKDRVDCGYPHVTPKECNNRGCCFDSRIPGVPWCF | 120 | |
| hTFF1 | 14 | LVSML-ALGTLA\|EAQTET----C-TVAPRERQNCGFPGVTPSQCANKGCCFDDTVRGVPWCF | 69 | |
| hTFF2a | 13 | LVLGLCALAGS-\|EKPSPCQ---CSRLSPHNRTNCGFPGITSDQCFDNGCCFDSSVTGVPWCF | 70 | |
| hTFF2b | 74 | ----------------PKQESDQC-VMEVSDRRNCGYPGISPEECASRKCCFSNFIFEVPWCF | 119 | |
| | | F | | |
| hTFF3 | 121 | KP-----LQEAECTF | 130 | |
| hTFF1 | 70 | YPNTIDVPPEEECEF | 84 | |
| hTFF2a | 71 | HPL------------ | 73 | |
| hTFF2b | 120 | FP-----KSVEDCHY | 129 | |

In the above alignment, SEQ ID NO:22 refers to the full-length human TFF3 sequence; SEQ ID NO:33 refers to the full-length human TFF1 sequence; SEQ ID NO:34 refers to amino acid residues 1-73 of full-length human TFF2 sequence (TFF2a) and SEQ ID NO:35 refers to amino acid residues of 74-129 of full-length human TFF2 sequence (TFF2b).

### Point Mutations are Determined as Follows:

TFF3 mature form:

(SEQ ID NO: 25)

EEYVGLSANQCAVPAKDRVDCGYPHVTPKECNNRGCCFDSRIPGVPWCFK
PLQEAECTF
TFF3 mutations: P24R, H25R, P46W47RR and C57F
TFF1 mature form:

(SEQ ID NO: 36)

EAQTETCTVAPRERQNCGFPGVTPSQCANKGCCFDDTVRGVPWCFYPNTI
DVPPEEECEF
TFF1 mutations: P20, G21R, P42W43RR and C58F,
TFF2 mature form:

(SEQ ID NO: 37)

KPSPCQCSRLSPHNRTNCGFPGITSDQCFDNGCCFDSSVTGVPWCFHPLP
KQESDQCVMEVSDRRNCGYPGISPEECASRKCCFSNFIFEVPWCFFPKSV
EDCHY
TFF2a: P21R, G22R, P43W44RR
TFF2b: P70R, G71R, P92W93RR and C103F

TFF antagonists also include fragments of the wild type full length sequence. For example, the following fragments were made by deleting portions of the protein, i.e., sequential removal of the C residues in TFF3. Corresponding TFF1 and TFF2 are generated in the same manner.

### TFF3-Deletion-1: N1-20

### TFF3-Deletion-2: N1-30

### TFF3-Deletion-3: N1-35

### TFF3-Deletion-4: N1-36

### TFF3-Deletion-5: N1-47

### TFF3-Deletion-6: N1-56

### TFF1-Deletion-1: N1-16

### TFF1-Deletion-2: N1-26

### TFF1-Deletion-3: N1-31

### TFF1-Deletion-4: N1-32

### TFF1-Deletion-5: N1-43

### TFF1-Deletion-6: N1-57

TFF2a-Deletion-1: N1-17
TFF2a-Deletion-2: N1-27
TFF2a-Deletion-3: N1-32
TFF2a-Deletion-4: N1-33
TFF2a-Deletion-5: N1-44
TFF2b-Deletion-1: N1-66
TFF2b-Deletion-2: N1-76
TFF2b-Deletion-3: N1-81
TFF2b-Deletion-4: N1-82
TFF2b-Deletion-5: N1-93
TFF2b-Deletion-6: N1-102

The domain and loop structure of TFF1, 2, and 3 are further defined in the following annotated depiction of the amino acid sequence of each protein. The mature form human TFF3 sequence disclosed in the following alignment is referred to as SEQ ID NO:25. The mature form human TFF1 sequence disclosed in the following alignment is referred to as SEQ ID NO:36. The mature form human TFF2 sequence disclosed in the following alignment is referred to as SEQ ID NO:37. The mature form porcine TFF2 sequence disclosed in the following alignment is referred to as SEQ ID NO:38.

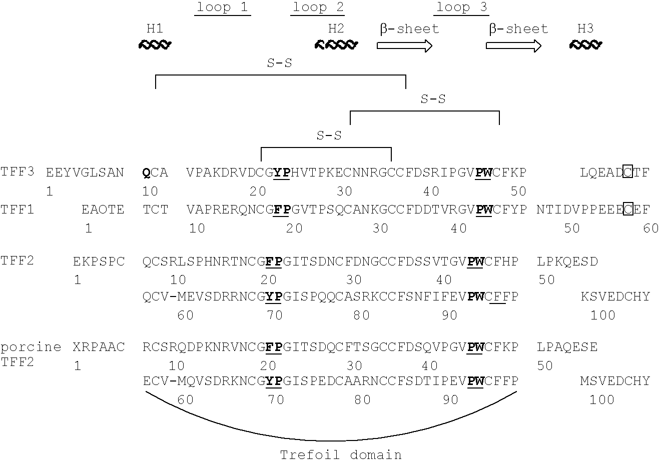

Peptide antagonists include peptides which may have been chemically modified. Such chemical modification increase stability in vivo or mimic natural post translational modifications. For example, peptides may be modified by acetylation, glycosylation, cross-linking, disulfide bond formation, cyclization, branching, phosphorylation, conjugation or attachment to a desirable molecule (for example conjugation to bispecific antibodies), acylation, ADP-ribosylation, amidation, covalent attachment of a lipid or lipid derivative, covalent attachment of phosphotidylinositol, demethylation, formation of covalent cross-links, formation of cysteine, formation of pyroglutamate, formylation, gamma-carboxylation, GPI anchor formation, hydroxylation, methylation, myristoylation, oxidation, pegylation, proteolytic processing, prenylation, racemization, sulfation, or otherwise to mimic natural post-translational modifications, for example. In addition, the peptides may be modified to include one or more non-naturally occurring amino acids. A peptide may be constructed of L or D amino acids as may be appropriate. Peptides of use in the invention may be modified to allow for targeting to specific cells or cell membranes. Fusion proteins are also included. Other suitable modifications known in the art are within the scope of the invention.

Optionally, a peptide antagonist is fused with, or otherwise incorporates, a motif which renders it cell-permeable (a cell membrane translocating motif). Such a motif is preferably a peptide-based membrane translocating motif. However, motifs of an alternative nature which may effectively provide cell-permeability; for example, motifs that are bound by and internalized by cell-surface receptors, or lipid moieties are within the scope of the invention. The Chariot transfection reagent is designed to transmit biologically active proteins and peptides into living cells, for example.

A peptide-based membrane translocating motif in accordance with the invention renders a peptide cell-permeable, whilst retaining at least a degree of the desired function of said peptide. Appropriate peptide-based membrane translocating motifs known in the art are within the scope of the invention. Preferred motifs include Penetratin and polymers of arginine. Further suitable peptide-based membrane translocating motifs are described in a review by Joliot and Prochiantz, Nat Cell Biol. 6(3):189-96, 2004.

Peptide antagonists also include chimeric peptides in which a suitable TFF peptide antagonist is fused or otherwise combined with another protein. By way of example, the invention provides chimeric peptides including a growth hormone receptor, receptor antagonist, angiostatin, endostatin, and thrombospondin A.

The invention also includes mimetics of the peptide antagonists of the invention. For example, peptides of the invention may be reduced to peptidomimetics whereby amino acid groupings from the peptide are arrayed on a scaffold, or further reduced to an entirely chemical small molecule inhibitor, or mix. Any suitable mimetic known in the art is within the scope of the invention “Mimetics” of the peptides of the invention will retain at least a degree of the desired function of said peptides.

Peptide antagonists of use in the invention may be made according to techniques and methodologies known in the art to which the invention relates, having regard to the published details of TFF nucleic acid and amino acid sequence information, and the teachings herein. By way of example, peptide antagonists may be made by chemical synthesis or using recombinant techniques.

Techniques for chemical synthesis of peptides include “solid phase” chemical synthesis carried out by FMOC chemistry as described in Fields G B, Lauer-Fields J L, Liu R Q and Barany G (2002) Principles and Practice of Solid-Phase peptide Synthesis; Grant G (2002) Evaluation of the Synthetic Product. Synthetic Peptides, A User's Guide, Grant GA, Second Edition, 93-219; 220-291, Oxford University Press, New York). However, any appropriate technique known in the art may be utilized.

Recombinant production of peptides of use in the invention will generally involve cloning and expression of TFF in host cells using appropriate nucleic acid vectors or constructs. Those of general skill in the art to which the invention relates will readily be able to identify nucleic acids which encode peptides of the invention, including desired fusion or chimeric peptides or proteins, on the basis of published TFF sequence data, or knowledge of a desired amino acid sequence for a peptide, the genetic code, and the understood degeneracy therein.

Nucleic acid constructs in accordance with the invention will generally contain nucleic acids encoding the desired peptide along with heterologous nucleic acid sequences; that is nucleic acid sequences that are not naturally found adjacent to the nucleic acid sequences of the invention. The constructs or vectors may be either RNA or DNA, either prokaryotic or eukaryotic, and typically are viruses or a plasmid. Suitable constructs are preferably adapted to deliver a nucleic acid of the invention into a host cell and are capable of replicating in such cell.

Constructs of use in cloning and expressing a peptide antagonist of the invention may contain regulatory sequences such as promoters, operators, repressors, enhancers, termination sequences, and other appropriate regulatory sequences as are known in the art. Further, the constructs may contain secretory sequences to enable an expressed peptide to be secreted from its host cell. In addition, the expression vectors may contain fusion sequences (such as those that encode a heterologous amino acid motif, for example Ubiquitin (which may aid in purification) or the likes of a peptide membrane-translocating motif as described elsewhere herein) which lead to the expression of inserted nucleic acid sequences of the invention as fusion proteins or peptides. Any suitable constructs or vectors known in the art are within the scope of the present invention.

A recombinant construct or vector comprising in accordance with the invention may be generated via recombinant techniques readily known to those of ordinary skill in the art to which the invention relates. For example, see Sambrook et al, Molecular Cloning: A Laboratory Manual (Third Edition) Cold Spring Harbor Laboratory Press, New York, USA.

In accordance with the invention, transformation of a construct into a host cell can be accomplished by any method by which a nucleic acid sequence can be inserted into a cell. For example, transformation techniques include transfection, electroporation, microinjection, lipofection, and adsorption.

As will be appreciated, transformed nucleic acid sequences may remain extrachromosomal or can integrate into one or more sites within a chromosome of a host cell in such a manner that their ability to be expressed is retained.

Any number of host cells known in the art may be utilized in cloning and expressing nucleic acid sequences of relevance to the invention. Host cells may be prokaryotic or eukaryotic. Examples of suitable host cells include Chinese Hamster Ovary (CHO) cells, insect cells (for example SF9 cells), and E. coli.

A recombinant peptide may be recovered from a transformed host cell, or culture media, following expression thereof using a variety of techniques standard in the art. For example, detergent extraction, osmotic shock treatment and inclusion body purification. The peptide may be further purified using techniques such as affinity chromatography, ion exchange chromatography, filtration, electrophoresis, hydrophobic interaction chromatography, gel filtration chromatography, and chromatofocusing.

A peptide of the invention may be in the form of a fusion peptide or protein; for example, a peptide of the invention attached to a peptide-based membrane translocating motif, or alternatively, or in addition, a motif which may aid in subsequent isolation and purification of the peptide (for example, ubiquitin, his-tag, or biotin). Means for generating such fusion peptides are readily known in the art to which the invention relates, and include chemical synthesis and techniques in which fusion peptides are expressed in recombinant host cells, as may be above mentioned. Strep-tag (Sigma-Genosys), Impact™ system (New England Biolabs), his-tag, and the eg pMAL™-p2 expression system (New England BioLabs), are particularly useful in the present invention. In addition, fusion tags of use in recombinant protein expression and purification have been described by R. C. Stevens. “Design of high-throughput methods of protein production for structural biology” Structure, 8, R177-R185 (2000).

Membrane translocating motifs may also be fused to a peptide by alternative means readily known in the art to which the invention relates. For example, where cell-permeabilising moieties comprise an entire protein, fatty acids and/or bile acids, such molecules may be linked to the active peptide by an amino acid bridge, or by a non-peptidyl linkage.

It will be appreciated that where a peptide antagonist of the invention is to include a mutation compared to the native TFF amino acid sequence (for example a single nucleotide insertion, deletion or substitution) various techniques may be employed. By way of example, molecular cloning techniques and site directed mutagenesis may be utilized. Persons of skill in the art to which the invention relates may readily appreciate alternative techniques; by way of example, see Sambrook et al, Molecular Cloning: A Laboratory Manual (Third Edition) Cold Spring Harbor Laboratory Press, New York, USA.

In as much as recombinant nucleic acid technology may be used to make a peptide antagonist of the invention, the invention also relates to nucleic acids encoding such peptides. In addition, while a peptide antagonist may be administered in the form of a peptide produced in vitro, it may be presented in the form of a nucleic acid adapted to express a peptide antagonist in use. Accordingly, the invention relates to nucleic acids and nucleic acid constructs adapted for the purposes of cloning or expression, including nucleic acid constructs adapted in use to produce a peptide antagonist of the invention.

The agent (e.g., peptides or nucleic acids of the invention) of use in inhibiting a TFF may be used on their own, or in the form of compositions in combination with one or more pharmaceutically acceptable diluents, carriers and/or excipients.

As-used herein, the phrase “pharmaceutically acceptable diluents, carriers and/or excipients” is intended to include substances that are useful in preparing a pharmaceutical composition, may be co-administered with an agent in accordance with the invention while allowing same to perform its intended function, and are generally safe, non-toxic and neither biologically nor otherwise undesirable. Examples of pharmaceutically acceptable diluents, carriers and/or excipients include solutions, solvents, dispersion media, delay agents, emulsions and the like. Diluents, carriers and/or excipients may contain minor amounts of additives such as substances that enhance isotonicity and chemical stability.

A variety of pharmaceutically acceptable diluents, carriers and/or excipients known in the art may be employed in compositions of the invention. As will be appreciated, the choice of such diluents, carriers and/or excipients will be dictated to some extent by the nature of the agent to be used, the intended dosage form of the composition, and the mode of administration thereof. By way of example, in the case of administration of nucleic acids such as vectors adapted to express antisense or iRNA, suitable carriers include isotonic solutions, water, aqueous saline solution, aqueous dextrose solution, and the like.

In addition to standard diluents, carriers and/or excipients, a pharmaceutical composition of the invention may be formulated with additional constituents, or in such a manner, so as to enhance the activity of the agent or help protect the integrity of the agent. For example, the composition may further comprise adjuvants or constituents which provide protection against degradation, or decrease antigenicity of an agent, upon administration to a subject. Alternatively, the agent may be modified so as to allow for targeting to specific cells, tissues or tumors.

The peptides or nucleic acids of the invention may be formulated to incorporate a sustained-release system. Inasmuch as this is the case, compositions may include semi-permeable polymer matrices in the form of shaped articles, e.g., films, or microcapsules. Sustained-release matrices include polylactides (U.S. Pat. No. 3,773,919; EP 58,481), copolymers of L-glutamic acid and gamma-ethyl-L-glutamate (Sidman et al., Biopolymers: 22: 547-56, 1983), poly(2-hydroxyethyl methacrylate) (Langer et al., J. Biomed. Mater. Res. 15: 267, 1981), ethylene vinyl acetate (Langer et al., J. Biomed. Mater. Res. 15: 267, 1981), or poly-D-(−)-3-hydroxybutyric acid (EP 133,988).

The peptides or nucleic acids of the invention of the invention may also be formulated into liposomes. Liposomes containing the compound may be prepared using techniques known in the art to which the invention relates. By way of example see: DE 3,218,121, EP 52,322, EP 36,676, EP 88,046, EP 143,949, EP 142,641, Japanese Pat. Appln. 83-118008, U.S. Pat. Nos. 4,485,045 and 4,544,545, and EP 102,324. Ordinarily, the liposomes are of the small (from or about 200 to 800 Angstroms) unilamellar type in which the lipid content is greater than about 30 mol percent cholesterol, the selected proportion being adjusted for the most efficacious therapy.

The peptides or nucleic acids of the invention of use in the invention may also be PEGylated to increase their lifetime.

Additionally, it is contemplated that a composition in accordance with the invention may be formulated with other ingredients which may be of benefit to a subject in particular instances. For example, there may be benefit in incorporating, where appropriate, one or more anti-neoplastic agents. Examples of such agents include: alkylating agents (e.g., chlorambucil (Leukeran™); cyclophosphamide (Endoxan™, Cycloblastin™, Neosar™, Cyclophosphamide™); ifosfamide (Holoxan™, Ifex™, Mesnex™); thiotepa (Thioplex™, Thiotepa™)), antimetabolites/S-phase inhibitors (e.g., methotrexate sodium (Folex™, Abitrexate™, Edertrexate™); 5-fluorouracil (Efudix™, Efudex™), hydroxyurea (Droxia™, Hydroxyurea, Hydrea™), amsacrine, gemcitabine (Gemzar™), dacarbazine, thioguanine (Lanvis™)), antimetabolites/mitotic poisons (e.g., etoposide (Etopophos™, Etoposide, Toposar™), vinblastine (Velbe™, Velban™), vindestine (Eldesine™), vinorelbine (Navelbine™), paclitaxel (Taxol™)), antibiotic-type agents (e.g., doxorubicin (Rubex™), bleomycin (Blenoxane™), dactinomycin (Cosmegen™), daunorubicin (Cerubidin™), mitomycin (Mutamycin™)), hormonal agents (e.g., aminoglutethimide (Cytadren™); anastrozole (Arimidex™); estramustine (Estracyt™, Emcyt™); goserelin (Zoladex™); hexamethylmelanine (Hexamet™); letrozole (Femara™); anastrozole (Arimidex™); tamoxifen (Estroxyn™, Genox™, Novaldex™, Soltamox™, Tamofen™)) or any combination of any two or more anti-neoplastic agents (e.g., Adriamycin/5-fluorouracil/cyclophosphamide (FAC); cyclophosphamide/methotrexate/5-fluorouracil (CMF)). The peptides or nucleic acids of the invention may also be formulated with compounds and agents, other than those specifically mentioned herein, in accordance with accepted pharmaceutical practice.

As will be appreciated, in the case of administration of nucleic acids, they may be packaged into viral delivery systems, which viral systems may themselves be formulated into compositions as herein described. Persons of skill in the art to which the invention relates may appreciate a variety of suitable viral vectors and methods which may be employed to implement such vectors having regard to the nature of the invention described herein. However, by way of example, retroviral vectors, adenoviral vectors, and Adeno-associated virus (AAV) can be used.

In accordance with the mode of administration to be used, and the suitable pharmaceutical excipients, diluents and/or carriers mentioned herein before, compositions of the invention may be converted to customary dosage forms such as solutions, orally administrable liquids, injectable liquids, tablets, coated tablets, capsules, pills, granules, suppositories, trans-dermal patches, suspensions, emulsions, sustained release formulations, gels, aerosols, liposomes, powders and immunoliposomes. The dosage form chosen will reflect the mode of administration desired to be used, the disorder to be treated and the nature of the agent to be used. Particularly preferred dosage forms include orally administrable tablets, gels, pills, capsules, semisolids, powders, sustained release formulation, suspensions, elixirs, aerosols, ointments or solutions for topical administration, and injectable liquids.

Skilled persons will readily recognise appropriate dosage forms and formulation methods. The compositions can be prepared by contacting or mixing specific agents and ingredients with one another. Then, if necessary, the product is shaped into the desired formulation. By way of example, certain methods of formulating compositions may be found in references such as Gennaro AR: Remington: The Science and Practice of Pharmacy, 20th ed., Lippincott, Williams & Wilkins, 2000.

The amount of a peptide or nucleic acid of the invention in a composition can vary widely depending on the type of composition, size of a unit dosage, kind of carriers, diluents and/or excipients, and other factors well known to those of ordinary skill in the art. The final composition can comprise from 0.0001 percent by weight (% w) to 100% w of the actives of this invention, preferably 0.001% w to 10% w, with the remainder being any other active agents present and/or carrier(s), diluent(s) and/or excipient(s).

Administration of any of the agents or compositions of the invention can be by any means capable of delivering the desired activity (inhibition of TFF) to a target site within the body of a subject. A “target site” may be any site within the body which may have or be susceptible to a proliferative disorder, and may include one or more cells, tissues or a specific tumor.

For example, administration may include parenteral administration routes, systemic administration routes, oral and topical administration. For example, administration may be by way of injection, subcutaneous, intraorbital, ophthalmic, intraspinal, intracisternal, topical, infusion (using e.g. slow release devices or minipumps such as osmotic pumps or skin patches), implant, aerosol, inhalation, scarification, intraperitoneal, intracapsular, intramuscular, intratumoral, intranasal, oral, buccal, transdermal, pulmonary, rectal or vaginal. As will be appreciated, the administration route chosen may be dependent on the position of the target site within the body of a subject, as well as the nature of the agent or composition being used.

In the case of nucleic acids, they can be administered for example by infection using defective or attenuated retroviral or other viral vectors (U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (e.g., Wu and Wu, J. Biol. Chem. 262:4429-4432, 1987) (which can be used to target cell types specifically expressing the receptors), and the like. In addition, nucleic acid-ligand complexes can be formed in which the ligand comprises a fusogenic viral peptide to disrupt endosomes, allowing the nucleic acid molecules to avoid lysosomal degradation.

The nucleic acid molecules can be targeted in vivo for cell specific uptake and expression, by targeting a specific receptor, as described for example in WO 92/06180; WO 92/22635; WO 92/20316; WO 93/14188 and WO 93/20221. Alternatively, the nucleic acid molecules can be introduced intracellularly and incorporated within host cell DNA for expression, by homologous recombination (Koller and Smithies, Proc. Natl. Acad. Sci. USA 86:8932-8935, 1989; Zijlstra et al., Nature 342:435-438, 1989).

Cells into which a nucleic acid can be introduced for purposes of the present invention encompass any desired, available cell type. The appropriate cell type will depend on the nature of the disorder to be treated. However, by way of example the nucleic acid is introduced to any hyperproliferative or neoplastic cell.

As will be appreciated, the dose of a peptide or nucleic acid of the invention or composition administered, the period of administration, and the general administration regime may differ between subjects depending on such variables as the nature of the condition to be treated, severity of symptoms of a subject, the size of any tumour to be treated, the target site to be treated, the mode of administration chosen, and the age, sex and/or general health of a subject. Persons of general skill in the art to which the invention relates will readily appreciate or be able to determine appropriate administration regimes having regard to such factors, without any undue experimentation. Administration of an agent of the invention is in an amount necessary to at least partly attain a desired response.

It should be appreciated that administration may include a single daily dose or administration of a number of discrete divided doses as may be appropriate.

Administration regimes can combine different modes or routes of administration. For example, intratumoural injection and systemic administration can be combined.

It should be appreciated that a method of the invention may further comprise further steps such as the administration of additional agents or compositions which may be beneficial to a subject having regard to the condition to be treated. For example, other agents of use in treating proliferative disorders (such as the anti-neoplastic agents mentioned above) could be administered. It should be appreciated that such additional agents and compositions may be administered concurrently with the agents and compositions of the invention, or in a sequential manner (for example the additional agents or compositions could be administered before or after administration of the agents or compositions of the invention. It should be appreciated in relation to sequential delivery of agents or compositions, that sequential administration of one agent or composition after the other need not occur immediately, although this may be preferable. There may be a time delay between delivery of the agents or compositions. The period of the delay will depend on factors such as the condition to be treated and the nature of the compositions or agents to be delivered. However, by way of example, the delay period can be between several hours to several days or months.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

What is claimed is:

1. A method of increasing sensitivity of a drug-resistant tumor to a chemotherapeutic agent, comprising contacting said drug-resistant tumor with an antibody composition that binds to a TFF gene product.

2. The method of claim 1, wherein said TFF gene product is TFF1.

3. The method of claim 1, wherein said TFF gene product is TFF3.

4. The method of claim 1, wherein said antibody composition comprises and antibody that binds to TFF1 and an antibody that binds to TFF3.

5. The method of claim 1, wherein said drug-resistant tumor is resistant to tamoxifen.

6. The method of claim 1, wherein said drug-resistant tumor is a breast carcinoma.

7. The method of claim 1, wherein said chemotherapeutic agent is tamoxifen.

8. The method of claim 1, wherein said chemotherapeutic agent is a hormone antagonist.

9. The method of claim 8, wherein said hormone antagonist is an anti-estrogen compound.


SEQUENCE LISTING (46 sequences)

SEQ ID NO: 1 - DNA, 21 bp, Homo sapiens
aatggccaccatggagaacaa

SEQ ID NO: 2 - DNA, 21 bp, Homo sapiens
aaataagggctgctgtttcga

SEQ ID NO: 3 - DNA, 59 bp, Artificial Sequence
  chemically synthesized iRNA
nnnnaatggccaccatggagaacaattcaagagattgttctccatggtggccattnnnn

SEQ ID NO: 4 - DNA, 59 bp, Artificial Sequence
  chemically synthesized iRNA
nnnnaaataagggctgctgtttcgattcaagagatcgaaacagcagcccttatttnnnn

SEQ ID NO: 5 - DNA, 21 bp, Homo sapiens
aatcaccagtgaccagtgttt

SEQ ID NO: 6 - DNA, 21 bp, Homo sapiens
aatggatgctgtttcgactcc

SEQ ID NO: 7 - DNA, 59 bp, Artificial Sequence
  chemically synthesized iRNA
nnnnaatcaccagtgaccagtgtttttcaagagaaaacactggtcactggtgattnnnn

SEQ ID NO: 8 - DNA, 59 bp, Artificial Sequence
  chemically synthesized iRNA to TFF2
nnnnaatggatgctgtttcgactccttcaagagaggagtcgaaacagcatccattnnnn

SEQ ID NO: 9 - DNA, 19 bp, Homo sapiens
actaggaagacagaatgca

SEQ ID NO: 10 - DNA, 18 bp, Homo sapiens
actaggaagacagaatgc

SEQ ID NO: 11 - DNA, 19 bp, Homo sapiens
tgctttgactccaggatcc

SEQ ID NO: 12 - DNA, 56 bp, Artificial sequnece
  chemically synthesized iRNA for TFF3
nnnncactaggaagacagaatgcattcaagagatgcattctgtcttcctagtnnnn

SEQ ID NO: 13 - DNA, 53 bp, Artificial Sequence
  chemically synthesized iRNA for TFF3
nnnnactaggaagacagaatgcttcaagagagcattctgtcttcctagtnnnn

SEQ ID NO: 14 - DNA, 55 bp, Artificial Sequence
  chemically synthesized iRNA for TFF3
nnnntgctttgactccaggatccttcaagagaggatcctggagtcaaagcannnn

SEQ ID NO: 15 - DNA, 74 bp, Artificial Sequence
  chemically synthesized iRNA for TFF1
ggatcccaatggccaccatggagaacaattcaagagattgttctccatggtggccattttttttccaaaagctt

SEQ ID NO: 16 - DNA, 74 bp, Artificial Sequnece
  chemically synthesized iRNA for TFF1
ggatcccaaataagggctgctgtttcgattcaagagatcgaaacagcagcccttatttttttttccaaaagctt

SEQ ID NO: 17 - DNA, 74 bp, Artificial Sequence
  chemically synthesized iRNA for TFF2
ggatcccaatcaccagtgaccagtgtttttcaagagaaaacactggtcactggtgattttttttccaaaagctt

SEQ ID NO: 18 - DNA, 74 bp, Artificial Sequence
  chemically synthesized iRNA for TFF2
ggatcccaatggatgctgtttcgactccttcaagagaggagtcgaaacagcatccattttttttccaaaagctt

SEQ ID NO: 19 - DNA, 70 bp, Artificial Sequence
  chemically synthesized iRNA for TFF3
ggatcccactaggaagacagaatgcattcaagagatgcattctgtcttcctagtttttttccaaaagctt

SEQ ID NO: 20 - DNA, 68 bp, Artificial Sequence
  chemically synthesized iRNA for TFF3
ggatcccactaggaagacagaatgcttcaagagagcattctgtcttcctagtttttttccaaaagctt

SEQ ID NO: 21 - DNA, 71 bp, Artificial Sequence
  chemically synthesized iRNA for TFF3
ggatcccgtgctttgactccaggatccttcaagagaggatcctggagtcaaagcattttttccaaaagctt

SEQ ID NO: 22 - protein, 130 aa, Homo sapiens
MQERTGAATARRESLPQANNPEQLCKQRCINEASWTMKRVLSCVPEPTVVMAARALCMLGLVLALLSSSSAEEYVGLSANQCAVPAKDRVDCGYPHVTPKECNNRGCCFDSRIPGVPWCFKPLQEAECTF

SEQ ID NO: 23 - protein, 50 aa, Homo sapiens
MQERTGAATARRESLPQANNPEQLCKQRCINEASWTMKRVLSCVPEPTVV

SEQ ID NO: 24 - protein, 21 aa, Homo sapiens
MAARALCMLGLVLALLSSSSA

SEQ ID NO: 25 - protein, 59 aa, Homo sapiens
EEYVGLSANQCAVPAKDRVDCGYPHVTPKECNNRGCCFDSRIPGVPWCFKPLQEAECTF

SEQ ID NO: 26 - DNA, 24 bp, Artificial Sequence
  chemically synthesized TFF3 sense
ctgaggcacctccagctgcccccg

SEQ ID NO: 27 - DNA, 21 bp, Artificial Sequence
  chemically synthesized TFF3 antisense
ggagcatgggacctttattcg

SEQ ID NO: 28 - DNA, 21 bp, Artificial Sequence
  chemically synthesized TFF3 cDNA top primer
ctctgcatgctggggctggtc

SEQ ID NO: 29 - DNA, 24 bp, Artificial Sequence
  chemically synthesized TFF3 cDNA Bottom primer
ggaggtgcctcagaaggtgcattc

SEQ ID NO: 30 - DNA, 24 bp, Artificial Sequence
  chemically synthesized TFF3 cDNA myc Top primer
gcgaagcttatgctggggctggtc

SEQ ID NO: 31 - DNA, 24 bp, Artificial Sequence
  chemically synthesized TFF3 cDNA Myc Bottom primer
ggaggtccgcgggaaggtgcattc

SEQ ID NO: 32 - DNA, 19 bp, Artificial Sequence
  chemically synthesized iRNA for TFF3
actaggaagacagaatgca

SEQ ID NO: 33 - protein, 84 aa, Homo sapiens
MATMENKVICALVLVSMLALGTLAEAQTETCTVAPRERQNCGFPGVTPSQCANKGCCFDDTVRGVPWCFYPNTIDVPPEEECEF

SEQ ID NO: 34 - protein, 73 aa, Homo sapiens
MGRRDAQLLAALLVLGLCALAGSEKPSPCQCSRLSPHNRTNCGFPGITSDQCFDNGCCFDSSVTGVPWCFHPL

SEQ ID NO: 35 - protein, 56 aa, Homo sapiens
PKQESDQCVMEVSDRRNCGYPGISPEECASRKCCFSNFIFEVPWCFFPKSVEDCHY

SEQ ID NO: 36 - protein, 60 aa, Homo sapiens
EAQTETCTVAPRERQNCGFPGVTPSQCANKGCCFDDTVRGVPWCFYPNTIDVPPEEECEF

SEQ ID NO: 37 - protein, 105 aa, Homo sapiens
KPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHY

SEQ ID NO: 38 - protein, 105 aa, Sus scrofa
  misc_feature (1)..(1): Xaa can be any naturally occurring amino acid
XPAACRCSRQSPKNRVNCGFPGITSDQCFTSGCCFDSQVPGVPWCFKPLPAQESEECVMQVSDRKNCGYPGISPEDCAARNCCFSDTIPEVPWCFFPMSVEDCHY

SEQ ID NO: 39 - DNA, 21 bp, Artificial Sequence
  chemically synthesized TFF3 sense cDNA primer
atggctgccagagcgctctgc

SEQ ID NO: 40 - DNA, 23 bp, Artificial Sequence
  chemically synthesized TFF3 antisense cDNA primer
aaggtgcattctgcttcctgcag

SEQ ID NO: 41 - protein, 10 aa, Artificial Sequence
  chemically synthesized Myc tag
EQKLISEEDL

SEQ ID NO: 42 - DNA, 25 bp, Artificial Sequence
  chemically synthesized pIRESneo3-TFF3-C57F-Myc antisense primer
aaggtgaattctgcttcctgcaggg

SEQ ID NO: 43 - DNA, 24 bp, Artificial Sequence
  chemically synthesized pIRESneo3 vector sense primer
atgaagtgggtaacctttatttcc

SEQ ID NO: 44 - DNA, 22 bp, Artificial Sequence
  chemically synthesized pIRESneo3 vector antisense primer
aagcctaaggcagcttgacttg

SEQ ID NO: 45 - DNA, 22 bp, Artificial Sequence
  chemically synthesized MCF-7 cell sense primer
ccagcatagacactctctttgg

SEQ ID NO: 46 - DNA, 22 bp, Artificial Sequence
  chemically synthesized MCF-7 cell antisense primer
ggtctctgctgtcttctctgtc